(19) United States
(12) Patent Application Publication
(10) Pub. No.: US 2023/0141499 A1
(43) Pub. Date: May 11, 2023

(54) CARDIOSPHERE-DERIVED CELL (CDC) THERAPY FOR THE TREATMENT OF VIRAL INFECTIONS

(71) Applicants: Cedars-Sinai Medical Center, Los Angeles, CA (US); Capricor, Inc., Beverly Hills, CA (US)

(72) Inventors: Eduardo Marbán, Santa Monica, CA (US); Linda Marbán, Santa Monica, CA (US)

(21) Appl. No.: 17/906,464

(22) PCT Filed: Mar. 19, 2021
(86) PCT No.: PCT/US2021/023165

§ 371 Date: Sep. 15, 2022

Related U.S. Application Data
(60) Provisional application No. 62/992,711, filed on Mar. 20, 2020.

(51) Int. Cl. A61K 35/34; A61K 9/00; A61K 45/06

(52) U.S. Cl. CPC A61K 35/34; A61K 9/0019; A61K 45/06

(57) ABSTRACT

Several embodiments of the methods and compositions disclosed herein relate to methods of treating viral infections, such as those caused by coronaviruses. In some embodiments, CDCs are administered to a patient, to treat the viral infection. In some embodiments, CDC-derived exosomes are administered to a patient. In some embodiments combinations of CDCs and CDC-derived exosomes are used. In still additional embodiments, combination therapies, such as CDCs or CDC-derived exosomes in combination with another therapeutic, such as an anti-inflammotry or other immune modulator are used to treat viral infections. In some embodiments, the viral infection is COVID-19, which is caused by SARS-CoV-2.

### CROSS-REFERENCE TO RELATED APPLICATION

This application claims priority to U.S. Provisional Application No. 62/992,711, filed Mar. 20, 2020, the entirety of which is incorporated herein by reference.

### FIELD

Several embodiments disclosed herein relate to methods and compositions comprising cardiosphere-derived cells (“CDCs” or byproducts/derivatives from those cells, such as their exosomes) for treatment of viral infections, such as those caused by a coronavirus. In several embodiments, combinations of CDC (or CDC-exosomes) are administered to a subject having a viral infection, such as COVID-19. In several embodiments, the subjects receive a plurality of doses of a CDC-based therapy in order to reduce and/or eliminate sympotms of the infection.

### BACKGROUND

Infections, particualry viral infections, have the potential to run rampant due to the ability of the virus to be spread prior to a subject exhibiting symptoms of the infection. Once symptoms are evident, the subject may have already come into contact with, and thus spread, to virus to other individuals, or in some embodiments, onto surfaces. Particularly susceptible are patients with underlying conditions that make combating a viral infection more challenging. These include, but are not limited to, patients with underlying respiratory disease, immunocompromised patients or even those with a disease that affects an organ system that is adversely affected by viral infection, such as the cardiovascular system. Further, elderly patients may be at higher risk. The SARS-Cov2 virus is one such virus, and causes COVID-19. COVID-19 patients can develop symptoms, including, but not limited to cytokine storm, lymphocytopenia, acute respiratory distress syndrome, and various cardiac disease manisfestations including myocarditis, myocardial infarction and arrhythmias.

Even with the early stages of this infection, mortality in patients at the highest risk (elderly, with comorbidities including prior cardiorespiratory dysfunction) approaches 45-50%, even with state-of the art supportive care in an ICU. No treatment modality has been shown to reduce mortality and morbidity in critically-ill Covid patients. Given the pandemic nature of the illness, and its high morbidity and mortality, there is a compelling and urgent unmet medical need.

Embodiments disclosed herein relate to CDCs, CDC-derived exosomes and their use in treating viral infections, including, but not limited to COVID-19. In several embodiments, the CDCs and/or CDC-derived exosomes (and/or their use in combination with other medications) attenuate or offset a number of key abnormalities associated with high mortality in COVID-19 (or other viral infections), including elevations of Troponin I and IL6, while boosting regulatory T cell proliferation and activation, and beneficially modulating the phenotype and healing capacity of macrophages. In several embodiments, these effects mitigate the pathophysiology of SARS-Cov2 infection and yield improved outcomes for critically-ill Covid patients.

### SUMMARY

Provided herein is a method of treating a respiratory viral infection, comprising administering to a subject in need of treating a respiratory viral infection, or one or more symptoms or sequelae thereof, a therapeutically effective amount of cardiosphere-derived cells (CDCs) and/or CDC-derived exosomes, thereby treating the viral infection or one or more symptoms or sequelae thereof. Optionally, the respiratory viral infection is COVID-19. In some embodiments, the subject has severe COVID-19. In some embodiments, the subject has acute myocarditis, acute respiratory distress syndrome (ARDS) and/or hypoxemia. In some embodiments, the subject is on ventilatory support and/or is in an intensive care unit (ICU). Optionally, the subject is intubated. In some embodiments, the subject has elevated levels of one or more inflammatory markers or cytokines, and/or has lymphopenea. Optionally, the one or more inflammatory markers or cytokines comprise one or more of ferritin, CRP, IL-6, and TNFα.

In some embodiments, administering the therapeutically effective amount of CDCs and/or CDC-derived exosomes reduces a hyperinflammatory response, or sequelae thereof, associated with a SARS-CoV-2 infection. In some embodiments, administration of the therapeutically effective amount of CDCs and/or CDC-derived exosomes reduces one or more of respiratory distress, myocardial injury, and/or inflammatory cytokine level. In some embodiments, administration of the therapeutically effective amount of CDCs and/or CDC-derived exosomes reduces length of hospital stay, length of stay in an ICU, or duration of ventilatory support.

In some embodiments, the therapeutically effective amount of CDCs comprises from about 1×10⁷ to about 1×10⁹ cells. In some embodiments, the therapeutically effective amount of CDC-derived exosomes comprises from about 2×10⁸ to about 2×10¹⁰ exosomes.

In some embodiments, the method further comprises administering to the subject at least one additional dose of the therapeutically effective amount of cardiosphere-derived cells (CDCs) and/or CDC-derived exosomes. Optionally, the at least one additional dose is administered on about a weekly basis.

In some embodiments, the method further comprises administering to the subject at least one additional therapeutic, wherein the additional therapeutic is selected from an antiviral agent; an analgesic agent; anti-arthritic agent; anti-asthmatic agent; anticholinergic agent; antihistamine; anti-infective agent; anti-inflammatory agent; cardiovascular medicament; gastrointestinal medicament; immunosuppressive agent; leukotriene inhibitor; narcotic agonist or antagonist; peptide drug; phytonutrient; vasodilator; vitamins or mineral supplement, or a combination thereof. Optionally, the at least one additional therapeutic comprises one or more of: clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine, promethazine, diphenhydramine, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazine, terfenadine, chlorpheniramine, dimenhydrinate, fexofenadine, orphenadrine, pheniramine, cimetidine, famotidine, lafutidine, nizatidine, ranitidine, roxatidine, cortisone hydrocortisone, hydrocortisone-21-monoester, hydrocortisone-17,21-diesters, alclometasone, dexamethasone, flumethasone, prednisolone, methylprednisolone, betamethasone, fluocinonide, prednisone, triamcinolone, methotrexate, leflunomide, cyclophosphamide, azathioprine, celecoxib, rofecoxib, a soluble cytokine receptor, anti-cytokine antibody, etanercept, infliximab, cyclosporin, tacrolimus, and rapamycin. In some embodiments, the method further comprises administering to the subject an anti-viral therapy.

Also provided herein is a method of treating a respiratory viral infection, comprising administering to a subject in need of treating a respiratory viral infection, or one or more symptoms or sequelae thereof, a therapeutically effective amount of cardiosphere-derived cells (CDCs), thereby treating the respiratory viral infection or one or more symptoms or sequelae thereof, wherein the respiratory viral infection is COVID-19, wherein the therapeutically effective amount comprises from about 1×10⁷ to about 1×10⁹ CDCs, wherein administration of the therapeutically effective amount of CDCs reduces a hyperinflammatory response, or a sequelae thereof, associated with a SARS-CoV-2 infection.

Provided herein is a method of treating a respiratory viral infection, comprising: administering to a subject in need of treating a respiratory viral infection, or one or more symptoms or sequelae thereof, a population of cardiosphere-derived cells (CDCs) and/or CDC-derived exosomes, wherein the population comprises about 1×10⁸ to about 2×10⁸ CDCs and/or about 2×10⁸ to about 2×10¹⁰ CDC-derived exosomes; and administering to the subject at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof; wherein administering the CDCs and/or CDC-derived exosomes and the at least one additional therapeutic reduce one or more of respiratory distress, myocardial injury, and/or inflammatory cytokine level, thereby treating the respiratory viral infection or one or more symptoms or sequelae thereof. Optionally, the respiratory viral infection is COVID-19. Optionally, the subject has severe COVID-19. In some embodiments, the subject has acute respiratory distress syndrome (ARDS) and/or acute myocarditis. In some embodiments, the subject is on ventilatory support and/or is in an intensive care unit (ICU). In some embodiments, the subject has elevated levels of one or more inflammatory markers or cytokines, and/or has lymphopenea.

In some embodiments, the method further comprises administering to the subject at least one additional dose of the population of CDCs and/or CDC-derived exosomes. Optionally, the at least one additional dose is administered on about a weekly basis.

In some embodiments, the antihistamine is selected from clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine, promethazine, diphenhydramine, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazine, terfenadine, chlorpheniramine, dimenhydrinate, fexofenadine, orphenadrine, pheniramine, cimetidine, famotidine, lafutidine, nizatidine, ranitidine, and roxatidine, or a combination thereof. In some embodiments, the anti-inflammatory agent is a corticosteroid. In some embodiments, the anti-inflammatory agent is selected from cortisone hydrocortisone, hydrocortisone-21-monoester, hydrocortisone-17,21-diester, alclometasone, dexamethasone, flumethasone, prednisolone, methylprednisolone, betamethasone, fluocinonide, prednisone, triamcinolone, or a combination thereof. In some embodiments, the immunosuppressive agent is methotrexate, leflunomide, cyclophosphamide, azathioprine, or a combination thereof. In some embodiments, the cyclooxygenase-2 inhibitor is celecoxib, rofecoxib, or a combination thereof. In some embodiments, the cytokine inhibitor is a soluble cytokine receptor, anti-cytokine antibody, or a combination thereof. In some embodiments, the TNF-α inhibitor is etanercept, infliximab, or a combination thereof. In some embodiments, the additional therapeutic is cyclosporin, tacrolimus, rapamycin, or a combination thereof.

In some embodiments, the CDCs are allogeneic CDCs. In some embodiments, the exosomes are allogeneic CDC-derived exosomes.

In some embodiments, the administering comprises parenteral administration of the population of CDCs and/or CDC-derived exosomes. In some embodiments, the population of CDCs and/or CDC-derived exosomes is administered intravenously. In some embodiments, the administering comprises infusing or injecting the population of CDCs and/or CDC-derived exosomes. In some embodiments, the administering comprises administering the CDCs. In some embodiments, the administering comprises administering the CDC-derived exosomes.

In some embodiments, the subject has one or more comorbidities. In some embodiments, the subject has a cardiorespiratory dysfunction. In some embodiments, the subject has hypertension, diabetes, obesity, and/or coronary heart disease.

Also provided herein is a use of a therapeutically effective amount of CDCs and/or CDC-derived exosomes, and optionally at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof, for the treatment of a respiratory viral infection.

Also provided is a use of a therapeutically effective amount of CDCs and/or CDC-derived exosomes, and optionally at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof, in the manufacture of a medicament for the treatment of a respiratory viral infection.

In some embodiments, the respiratory viral infection is COVID-19. In some embodiments, the respiratory viral infection is severe COVID-19. In some embodiments, the therapeutically effective amount of CDCs comprises from about 1×10⁷ to about 1×10⁹ cells. In some embodiments, the therapeutically effective amount of CDC-derived exosomes comprises from about 2×10⁸ to about 2×10¹⁰ exosomes.

### DETAILED DESCRIPTION

Some embodiments of the methods and compositions provided herein relate to the use of CDCs, CDC-derived exosomes and/or traditional anti-inflammatory or immune modulating drugs to treat viral infections. One such viral infection is COVID-19. COVID-19 is caused by a coronavirus. Coronaviruses are a large family of viruses that are common in people and many different species of animals, including camels, cattle, cats, and bats. Rarely, animal coronaviruses can infect people and then spread between people such as with MERS-CoV, SARS-CoV, and now with this new virus (named SARS-CoV-2).

The SARS-CoV-2 virus is a betacoronavirus, like MERS-CoV and SARS-CoV. All three of these viruses have their origins in bats. The sequences from U.S. patients are similar to the one that China initially posted, suggesting a likely single, recent emergence of this virus from an animal reservoir.

The complete clinical picture with regard to COVID-19 is not fully known. Reported illnesses have ranged from very mild (including some with no reported symptoms) to severe, including illness resulting in death. Higher risk populations include older adults, with risk increasing by age as well as people who have serious chronic medical conditions including but not limited to heart disease, diabetes, and/or lung disease. However, as more is learned, even seemingly not-at-risk individuals can develop potentially life-threatening symptoms.

Described below are CDCs and CDC-derived exosomes, as well as the rationale for their use in methods of treating viral infections, including COVID-19.

Cardiospheres are undifferentiated cardiac cells that grow as self-adherent clusters. Briefly, heart tissue can be collected from a patient during surgery or cardiac biopsy. In some embodiments, heart tissue can be harvested from the left ventricle, right ventricle, septum, left atrium, right atrium, crista terminalis, right ventricular endocardium, septal or ventricle wall, atrial appendages, or combinations thereof. In some embodiments, a biopsy can be obtained, for example, using a percutaneous bioptome as described in US Pat. App. Pub. No. 2009/012422 and US Pat. App. Pub. No. 2012/0039857, the disclosures of which are herein incorporated by reference in their entireties. In some embodiments, the tissue can cultured directly, or alternatively, the heart tissue can be frozen, thawed, and then cultured. In some embodiments, the tissue can be digested with protease enzymes such as collagenase, trypsin and the like. In some embodiments, the heart tissue can be cultured as an explant such that cells including fibroblast-like cells and cardiosphere-forming cells grow out from the explant. In some embodiments, an explant is cultured on a culture vessel coated with one or more components of the extracellular matrix (e.g., fibronectin, laminin, collagen, elastin, or other extracellular matrix proteins). In some embodiments, the tissue explant can be cultured for about 1, 2, 3, 4, or more weeks prior to collecting the cardiosphere-forming cells. In some embodiments, a layer of fibroblast-like cells can grow from the explant onto which cardiosphere-forming cells appear. In some embodiments, cardiosphere-forming cells can appear as small, round, phase-bright cells under phase contrast microscopy. In some embodiments, cells surrounding the explant including cardiosphere-forming cells can be collected by manual methods or by enzymatic digestion. In some embodiments, the collected cardiosphere-forming cells can be cultured under conditions to promote the formation of cardiospheres. In some embodiments, the cells are cultured in cardiosphere-growth medium comprising buffered media, amino acids, nutrients, serum or serum replacement, growth factors including but not limited to EGF and bFGF, cytokines including but not limited to cardiotrophin, and other cardiosphere promoting factors such as but not limited to thrombin. In some embodiments, cardiosphere-forming cells can be plated at an appropriate density necessary for cardiosphere formation, such as about 20,000-100,000 cells/mL. In some embodiments, the cells can be cultured on sterile dishes coated with poly-D-lysine, or other natural or synthetic molecules that hinder the cells from attaching to the surface of the dish. In some embodiments, cardiospheres can appear spontaneously about 2-7 days or more after cardiosphere-forming cells are plated.

CDCs are a population of cells generated by manipulating cardiospheres. In some embodiments, CDCs can be generated by plating cardiospheres on a solid surface which is coated with a substance which encourages adherence of cells to a solid surface of a culture vessel (e.g., fibronectin, a hydrogel, a polymer, laminin, serum, collagen, gelatin, or poly-D-lysine) and expanding same as an adherent monolayer culture. In some embodiments, CDCs can be repeatedly passaged (e.g., passaged two times or more) according to standard cell culturing methods.

In various embodiments, the CDCs are generated from a biopsy sample cultured into an explant, further cultured into an explant derived cell, additionally cultured as cardiosphere forming cells, thereafter cultured as cardiospheres, and subsequently cultured as CDCs. In other embodiments, the CDCs are human.

In some embodiments, the CDCs are immortalized. In some embodiments, immortalized CDCs are used for deriving exosomes for use in the present methods or compositions. Advantageously, immortalized CDCs can be passaged more times than their non-immortalized counterpart. In some embodiments, immortalized CDCs are passaged 8 times or more, e.g., 9 times or more, 10 times or more 11 times or more, 12 times or more, 15 times or more, 18 times or more, 20 times or more, 25 times or more, 30 times or more, 40 times or more, including 50 times or more after the cardiosphere formation stage. Any suitable means of immortalizing CDCs may be used. Suitable means of generating high-potency exosomes, immortalized CDCs, and exosomes derived therefrom, are described, e.g., Ibrahim et al., Nat Biomed Eng. 2019 Sep;3(9):695-705, the entire disclosure of which is incorporated herein by reference.

Exosomes (or XOs) (e.g., CDC-derived exosomes) are vesicles formed via a specific intracellular pathway involving multivesicular bodies or endosomal-related regions of the plasma membrane of a cell. XOs can range in size from approximately 20-150 nm in diameter. Depending on the embodiment, the size of the exosomes ranges in diameter from about 15 nm to about 95 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In several embodiments, exosomes are larger (e.g., those ranging from about 140 to about 210 nm, including about 140 nm to about 150 nm, about 150 nm to about 160 nm, about 160 nm to about 170 nm, about 170 nm to about 180 nm, about 180 nm to about 190 nm, about 190 nm to about 200 nm, about 200 nm to about 210 nm, and overlapping ranges thereof). In some embodiments, the exosomes that are generated from the original cellular body are 100, 200, 300, 400, 500, 600, 700, 800, 900, 1000, 2000, 5000, or 10,000 times smaller in at least one dimension (e.g., diameter) than the original cellular body.

In some embodiments, XOs have a characteristic buoyant density of approximately 1.1-1.2 g/mL, and a characteristic lipid composition. In some embodiments, XOs can be isolated based on their size and buoyancy. XOs lipid membrane is typically rich in cholesterol and contains sphingomyelin, ceramide, lipid rafts and exposed phosphatidylserine. In some embodiments, XOs express certain marker proteins, such as integrins and cell adhesion molecules. In some embodiments, XOs generally lack markers of lysosomes, mitochondria, or caveolae. In some embodiments, XOs contain cell-derived components, such as but not limited to, proteins, DNA and RNA (e.g., microRNA and noncoding RNA). In some embodiments, XOs are isolated from other extracellular vesicles (EVs) and/or from cellular debris using one or more features of the XOs. In some embodiments, XOs can be obtained from cells obtained from a source that is allogeneic, autologous, xenogeneic, or syngeneic with respect to the recipient of the XOs.

In some embodiments, the exosomes, e.g., CDC-derived exosomes, of the present disclosure include a variety of biomolecules, such as nucleic acids and proteins. In some embodiments, the exosomes contain DNA, DNA fragments, DNA plasmids, mRNA, tRNA, snRNA, saRNA, miRNA, rRNA, regulating RNA, other non-coding and coding RNA, etc. In some embodiments, the exosomes contain non-coding RNAs (ncRNAs), such as, but not limited to, long non-coding RNAs (IncRNAs), microRNAs (miRNAs) and Y RNA fragments.

In some embodiments, the exosomes, e.g., CDC-derived exosomes, are enriched in or depleted for one or more biomolecules, such as nucleic acids and proteins. In some embodiments, a biomolecule may be enriched (or depleted) in the exosomes relative to the level of a suitable reference biomolecule. In some embodiments, exosomes, e.g., the CDC-derived exosomes, are enriched for a miRNA relative to a reference miRNA. In some embodiments, the exosomes, e.g., CDC-derived exosomes, are depleted for a miRNA relative to a reference miRNA. In some embodiments, an miRNA is enriched if the amount of miRNA present in the exosomes is 5, 10, 15, 20, 25, 30, 40, 50, 75, 100, 200, 300, 500, 750, 1,000, 2,000, 5,000, 10,000 fold or more, or has a fold change in a range defined by any two of the preceding values, than the amount of the reference miRNA present in the exosomes. In some embodiments, an miRNA is depleted if the amount of miRNA present in the exosomes is 0.75, 0.5, 0.4, 0.3, 0.2, 0.1, 0.08, 0.06, 0.04, 0.02, 0.01, 0.005, 0.001 fold or less, or has a fold change in a range defined by any two of the preceding values, than the amount of the reference miRNA present in the exosomes. In some embodiments, the CDC-derived exosomes are enriched or depleted for a biomolecule (e.g., miRNA) relative to the level of the biomolecule in non-therapeutic exosomes (e.g., exosomes derived from human dermal fibroblasts (HDFs)).

In some embodiments, certain types of RNA, e.g., microRNA (mi RNA), are enriched or are carried by XOs. In some embodiments, miRNAs function as post-transcriptional regulators, often through binding to complementary sequences on target messenger RNA transcripts (mRNAs), thereby resulting in translational repression, target mRNA degradation and/or gene silencing. In some embodiments, miR146a exhibits over a 250-fold increased expression in XOs from CDCs, and miR210 is upregulated approximately 30-fold in XOs from CDCs, as compared to the XOs isolated from normal human dermal fibroblasts.

In some embodiments, the exosomes, e.g., CDC-derived exosomes, contain or are enriched for miR-92a, miR-181b, miR-148a, and/or miR-146a. In some embodiments, the exosomes, e.g., CDC-derived exosomes, do not contain, or are depleted for miR-199b. In some embodiments, the exosomes are enriched for miR-92a, miR-181b, miR-148a, and/or miR-146a relative to the level of miR-199b in the exosomes. In some embodiments, the exosomes are enriched for miR-92a, miR-181b, miR-148a, and/or miR-146a relative to a reference miRNA, e.g., miR-23a. In some embodiments, the exosomes are depleted for miR-199b relative to a reference miRNA, e.g., miR-23a.

Methods for preparing XOs can include (or lack) one or more of the following steps: culturing cardiospheres or CDCs in conditioned media, isolating the cells from the conditioned media, purifying the exosome (e.g., by sequential centrifugation, etc.), optionally, clarifying the XOs on a density gradient (e.g., a sucrose density gradient). In some embodiments, the isolated and purified XOs are essentially free of non-exosome components, such as components of cardiospheres or CDCs. In some embodiments, XOs can be resuspended in a buffer such as a sterile PBS buffer containing 0.01-30%, 0.01-0.1%, 0.1-1%, 1-10%, 10-20%, or 20-30% human serum albumin. In some embodiments, the XOs may be frozen and stored for future use.

In some embodiments, XOs can be prepared using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. In some embodiments, methods for isolating XOs from stem cells are found in US Pat. App. Pub. No. 2012/0093885 and US Pat. App. Pub. No. 2014/0004601, which are hereby incorporated by reference. In some embodiments, collected XOs can be concentrated and/or purified using methods as disclosed elsewhere herein. In some embodiments, specific methodologies include, but are not limited to, ultracentrifugation, density gradient, HPLC, adherence to substrate based on affinity, or filtration based on size exclusion.

In some embodiments, for example, differential ultracentrifugation is used to isolate secreted XOs from the supernatants of cultured cells. In some embodiments, this approach allows for separation of XOs from nonmembranous particles, by exploiting their relatively low buoyant density. In some embodiments, size exclusion allows for their separation from biochemically similar, but biophysically different microvesicles (MVs), which possess larger diameters of (e.g., up to 1,000 nm). In some embodiments, differences in flotation velocity further allows for separation of differentially sized XOs. In some embodiments, XOs sizes will possess a diameter ranging from 30-200 nm, including sizes of 40-100 nm. In some embodiments, purification may rely on specific properties of the particular XOs of interest. In some embodiments, this includes, e.g., use of immunoadsorption with a protein of interest to select specific vesicles with exoplasmic or outward orientations. In some embodiments, more than one purification technique can be used together.

In some embodiments, one or more of differential centrifugation, discontinuous density gradients, immunoaffinity, ultrafiltration and high performance liquid chromatography (HPLC), differential ultracentrifugation can be used for exosome isolation. In some embodiments, the centrifugation technique utilizes increasing centrifugal force from 2000×g to 10,000×g to separate the medium- and larger-sized particles and cell debris from the exosome pellet at 100,000×g. In some embodiments, centrifugation alone allows for significant separation/collection of XOs from a conditioned medium. In some embodiments, enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2g/mL) or application of a discrete sugar cushion in preparation.

In some embodiments, ultrafiltration can be used to purify XOs without compromising their biological activity. In some embodiments, membranes with different pore sizes—such as 100 kDa molecular weight cut-off (MWCO) and gel filtration to eliminate smaller particles—have been used to avoid the use of a non-neutral pH or non-physiological salt concentration. In some embodiments, tangential flow filtration (TFF) systems are scalable (to >10,000L), allowing one to not only purify, but concentrate the exosome fractions, and such approaches are less time consuming than differential centrifugation. HPLC can also be used to purify XOs to homogeneously sized particles and preserve their biological activity as the preparation is maintained at a physiological pH and salt concentration.

In some embodiments, other chemical methods exploit differential solubility of XOs for precipitation techniques, addition to volume-excluding polymers (e.g., polyethylene glycols (PEGs)), possibly combined additional rounds of centrifugation or filtration. In some embodiments, for example, a precipitation reagent, ExoQuick®, can be added to conditioned cell media to quickly and rapidly precipitate a population of XOs. In some embodiments, flow field-flow fractionation (FIFFF) is an elution-based technique that is used to separate and characterize macromolecules (e.g., proteins) and nano- to micro-sized particles (e.g., organelles and cells) and which can be successfully applied to fractionate XOs from culture media.

In some embodiments, focused techniques may be applied to isolate specific XOs of interest. In some embodiments, this includes relying on antibody immunoaffinity to recognizing certain exosome-associated antigens. In some embodiments, XOs further express the extracellular domain of membrane-bound receptors at the surface of the membrane. In some embodiments, this presents a ripe opportunity for isolating and segregating XOs in connections with their parental cellular origin, based on a shared antigenic profile. In some embodiments, conjugation to magnetic beads, chromatography matrices, plates or microfluidic devices allows isolating of specific exosome populations of interest as may be related to their production from a parent cell of interest or associated cellular regulatory state. In some embodiments, other affinity-capture methods use lectins which bind to specific saccharide residues on the exosome surface.

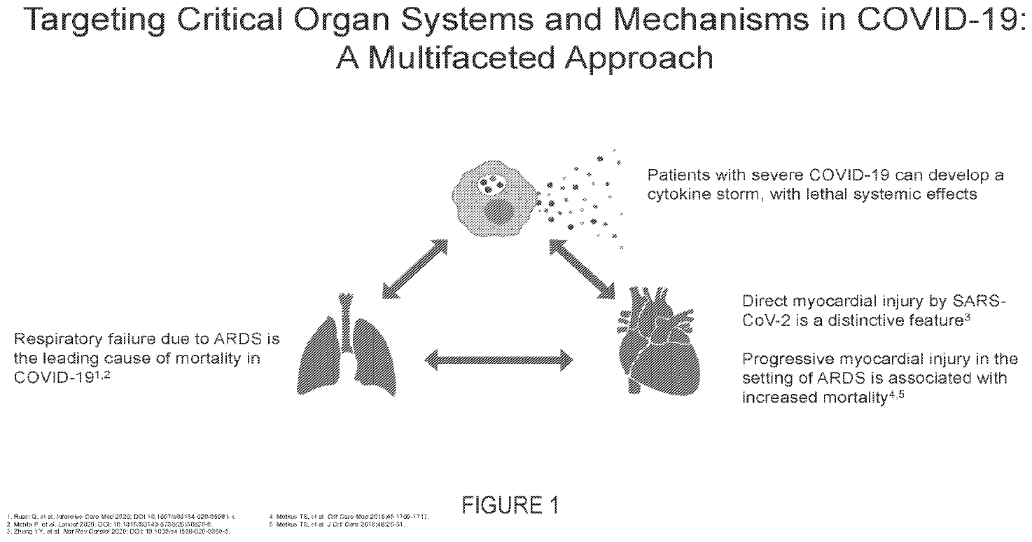

FIG. 1 shows a schematic depiction of the interplay of various symptoms and organ systems that are impacted during COVID-19 infection. Cytokine storm, respiratory distress and direct myocardial injury are each alone, and in combination, responsible for increased mortality. In several embodiments, the use of CDCs and/or CDC-derived exosomes can reduce, eliminate, or otherwise ameliorate one or more of those symptoms (among other disclosed herein) to improve the patient outcomes for patients with viral infections, such as COVID-19.

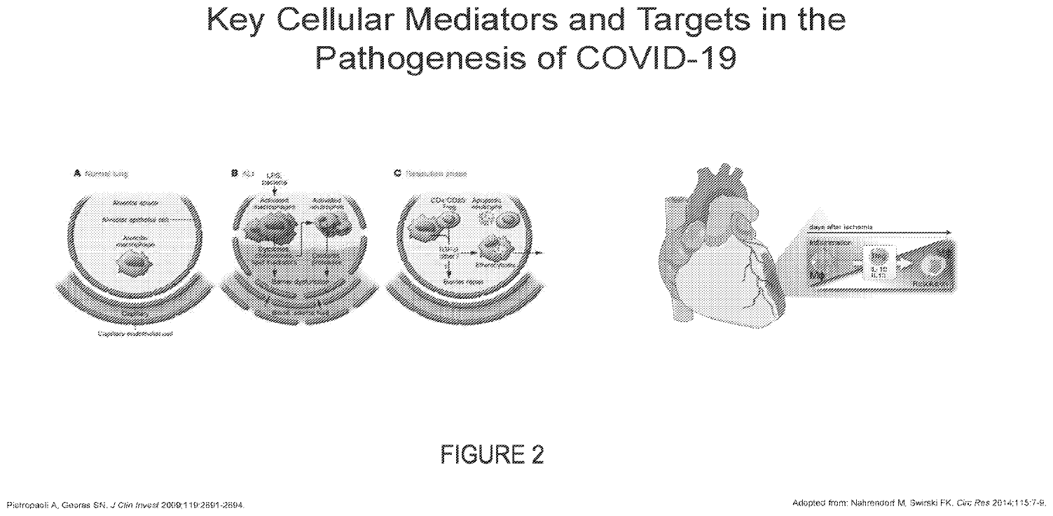

FIG. 2 schematically depicts some of cellular mediators believed to be key to pathogenesis of viral infection, in particular COVID-19. The left panel of FIG. 2 depicts a normal lung, where the alveolar epithelium is intact. Infection, for example with bacteria or viruses, results in disruption of the epithelial cell barrier, due in part to the activation of macrophages and/or neturophils. Activated macrophages release cytokines, lipid mediators and/or chemokines that disrupt the epithelial barrier. Likewise, activated neutrophils release various oxidants or proteases, also disrupting alveolar epithelial cells. The Part C of the left panel shows how activation of Treg cells and release of TGFbeta (and perhaps other mediators) can result in barrier repair as well as promote the the efferocytosis of apoptotic neutrophils. The right panel of FIG. 2 shows mediators of cardiac tissue damage after an ischemic event. These include macrophoage infiltration and inflammation. Treg cells and release of IL10, IL13, and potentially other mediators lead to resolution.

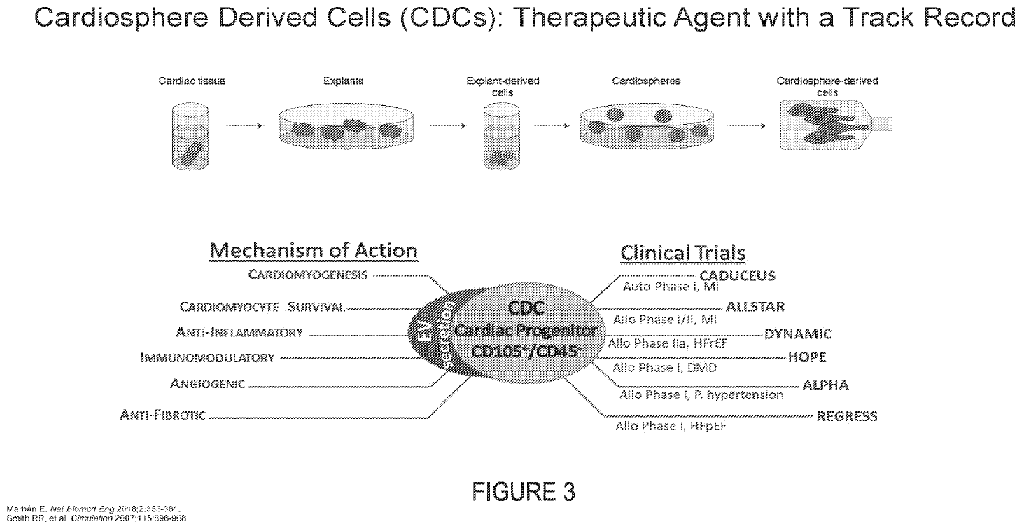

FIG. 3 shows a schematic depiction of CDC production, which is described in more detail elsewhere herein. The lower portion of FIG. 3 shows a schematic related to prior studies using CDCs as well as their mechanisms of action, which include, but are not limited to cardiomyogenesis, enhancing cardiomyocyte survival, anti-inflammatory effects, immunomodulatory effects, angiogenic effects, and/or anti-fibrotic effects.

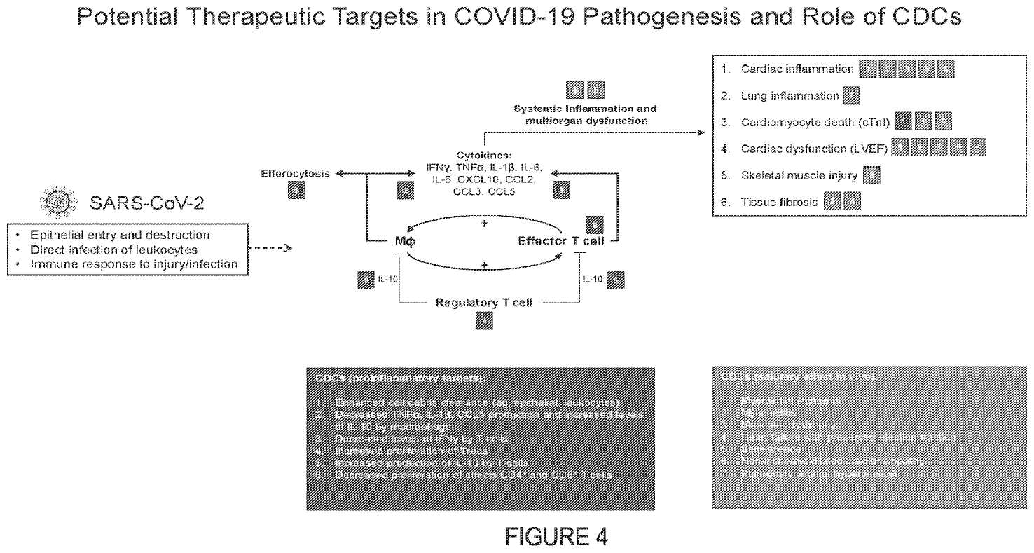

FIG. 4 shows a schematic of potential therapeutic targets in viral infection, particularly COVID-19, as well as selected salutary effects of CDCs that may offset the pathogenesis. CDCs exhibit, according to several embodiments, various beneifical effects by targeting and reducing pro-inflammatory symptoms. For example, CDCs promote enhanced clearance of cellular debris that can increase the efferocytosis of cells, for example alveolar cells, which can promote resolution of lung infection, reduce respiratory distress and promote alveolar epithelial cell recovery. CDCs can also reduce secretion of TNFalpha, IL-1beta, CCL5 and other cytokines that can lead to systemic inflammation. Likewise IL10 secretion by macrophages is upregulated (also in T cells), which promotes proliferation and activity of Treg cells. CDCs can reduce TNF alpha production by T cells, which helps reduce systemic inflammation. CDCs can also reduce the proliferation of CD4 and CD8 positive T cells, which can further reduce proimflammatory effects caused by active T cells. Additionally, CDCs exhibit at least the indicated salutary effects in vivo as well, which can further improve patient outcomes.

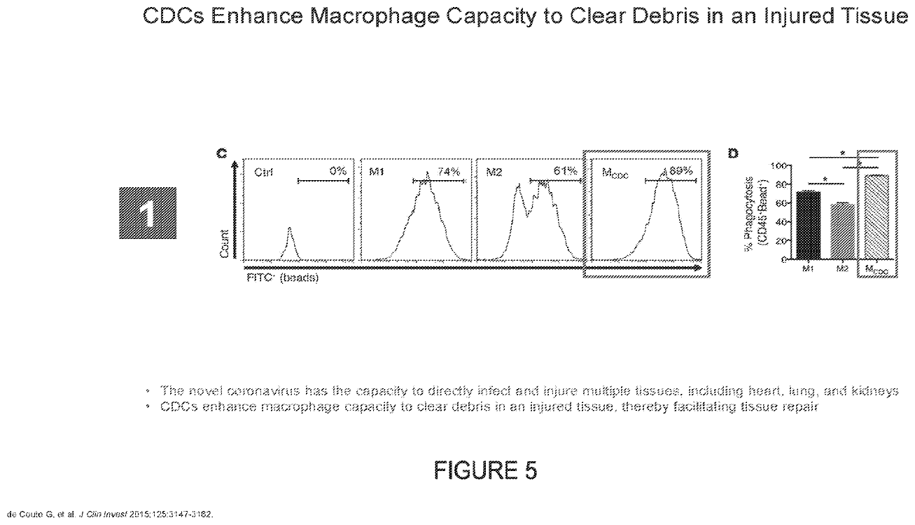

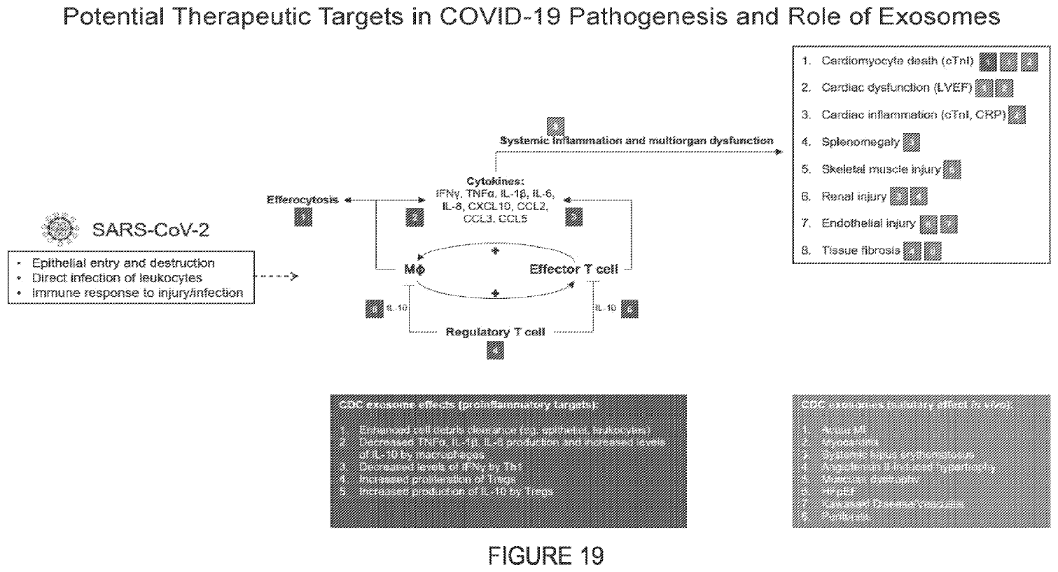

In the Figures, the large numbered box corresponds to a point of action in the schematic of disease pathogenesis (shown in FIG. 4 for CDCs and FIG. 19 for CDC-derived exosomes). FIG. 5 shows data related to the enhanced ability of macrophages to clear cellular debris in injured tissue upon administration of CDCs. According to some embodiments, this enhanced clearance reduces the impact/damage that viral infections have on multiple tissues, including heart, lung and kidneys.

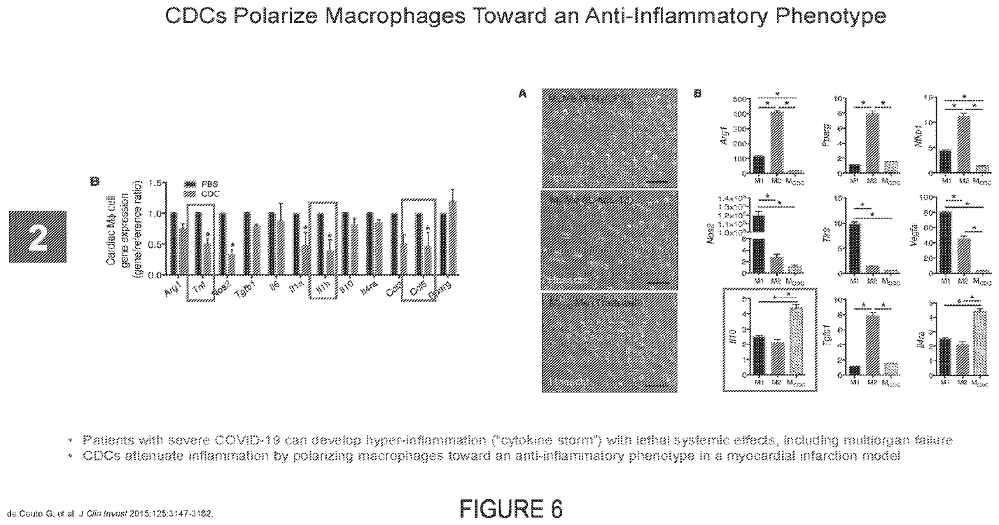

FIG. 6 shows data related to the ability of CDCs to polarize macrophages to an anti-inflammatory phenotype. COVID-19 patients can develop what is known as cytokine storm, which can impact multiple organ systems, potentially leading to multiorgan failure. In several embodiments, the use of CDCs can attenuate inflammation by polarizing macrophages towards an anti-inflammatory phenotype. FIG. 6 shows this data in the context of a myocardial infarction model. The boxed histogram bars on the left show the reduction of TNFalpha, IL1beta, and CCLS exprssion on macrophages after CDC administration. The right panel shows increased IL10 production by macrophages after CDC exposure.

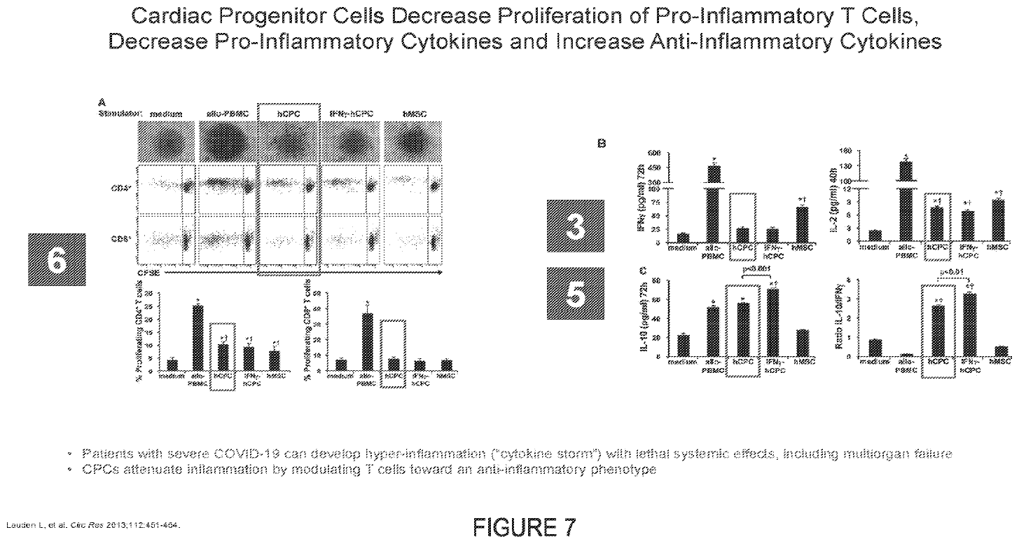

FIG. 7 shows data related to the ability of CDCs to decrease proliferation of proinflammatory T cells, decrease proinflammatory cytokines and increase anti-inflammatory cytokines. As discussed above, patients with severe COVID-19 can develop hyperinflammation (cytokine storm). The left panel of FIG. 7 shows that CDCs (here labeled as human cardiac progenitor cells, hCPC) reduce the proliferation of both CD4+and CD8+T cells. The right panels show data related to the reduction of release of pro-inflammatory cytokines by T cells when CDCs are administered. Shown in the top panels are data related to reduced interferon gamma and IL2 release by T cells. The bottom two panels show increase production of IL10 by T cells and a a shift in the ratio of IL10:IFNg release, representing a shift in T cells towards an anti-inflammatory phenotype.

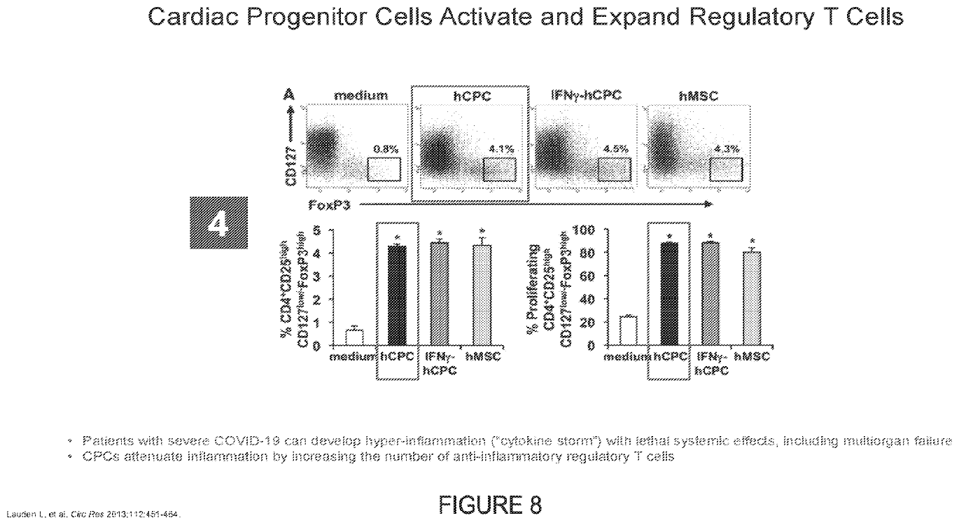

FIG. 8 shows that CDCs (labled as hCPC) can activate and expand regulatoy T cells. The top row of data shows that coculturing of CD4+T cells with hCPC induces a subpopulation of T-regulatory phenotype. Quantification indicates that hCPC induce an increase of CD4+CD25highCD127low/-FoxP3high cells with at least 80% of these cells being proliferative, as shown in the bottom 2 panels. Thus, CDC administration can attenuate inflammation by increasing the number of anti-inflammatory regulatory T cells.

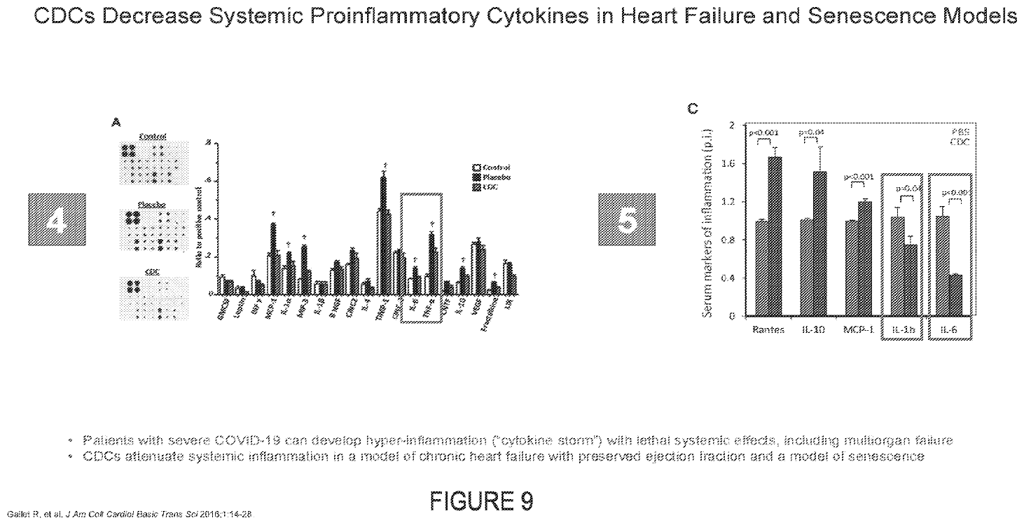

FIG. 9 shows data related to the ability of CDCs to decrease systemic proinflammatory cytokines in heart failure (panel A) and senescence models (panel C). Panel A shows that CDC treatment normalizes the expression of proinflammatory cytokines, IL-6 and TNF-a, in a rat model of heart failure with preserved ejection fraction (HFpEF). Similarly, in Panel C, reduction of serum markers of inflammation, IL-1b and IL-6, can be seen in CDC-treated rat model of senescence. These effects of CDCs, in several embodiments, attenuate systemic inflammation and help prevent cytokine storm in virally infected patients.

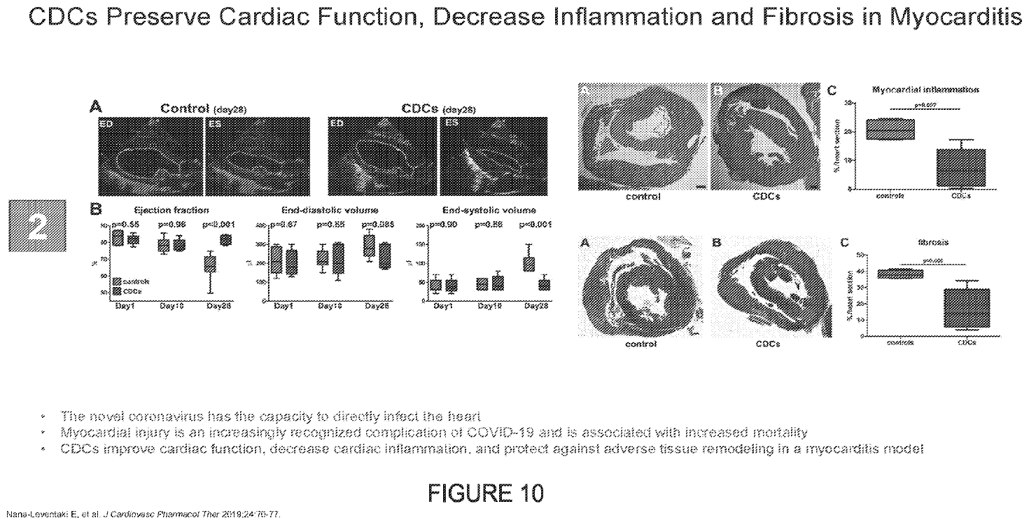

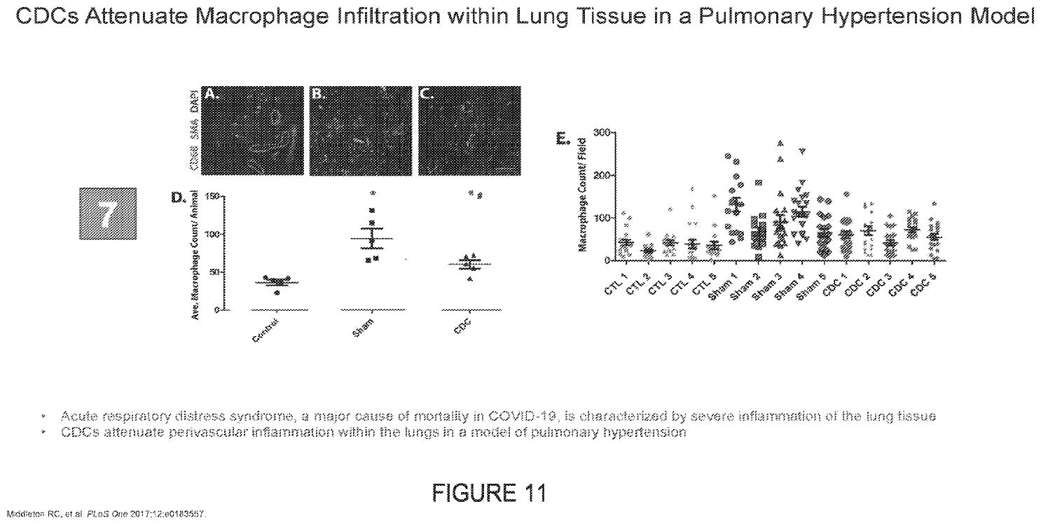

FIG. 10 shows data related to the ability of CDCs to preserve cardiac function, decrease inflammation and fibrosis in myocarditis. As discussed above, SARS-CoV-2 has the ability to directly infect the heart and myocardial injury is increasingly being observed as a complication of COVID-19. The left panel shows echocardiography data from control and CDC treated hearts. The lower panel shows data indicating that CDC administration preserves ejection fraction and other aspects of cardiac function. The staining data in the right panels show that CDC administration reduces both myocardial inflammation (top row) and myocardial fibrosis (lower row). CDCs, with these beneficial effects on heart function and structure may help reduce the pathogenesis of COVID-19, or other viral infections.

Respiratory distress, specifically acute respiratory distress syndrome in COVID-19, is a major cause of mortality, and is characterized by severe inflammation of lung tissue. CDCs have been demonstrated to reduce macrophage infiltration into the lung in a pulmonary hypertension model. The left panel of FIG. 11 shows immunohistochemistry and summary data shown in that CDC administration lowers the avergage macrophage count in animal lungs during pulmonary hypertension as compared to sham. The right panel shows reduced macrophage count per micorscopic field of view. Thus, CDCs can beneificialyl reduce the propensity for lung damage during disease, including in several embodiments COVID-19.

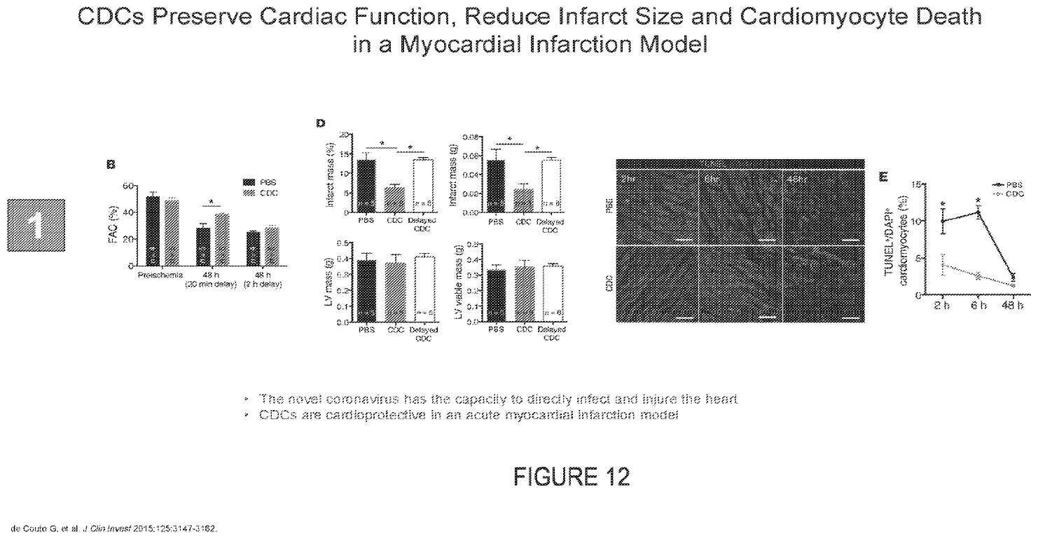

FIG. 12 shows data related to the ability of CDCs to preserve cardiac function, reduce infarct size and reduce cardiomyocyte death in an acute myocardial infarction model. These cardioprotective effects, in several embodiments, aid in reducing the pathogenesis of COVID-19, particularly the damage the SARS-CoV-2 virus can induce directly in the heart.

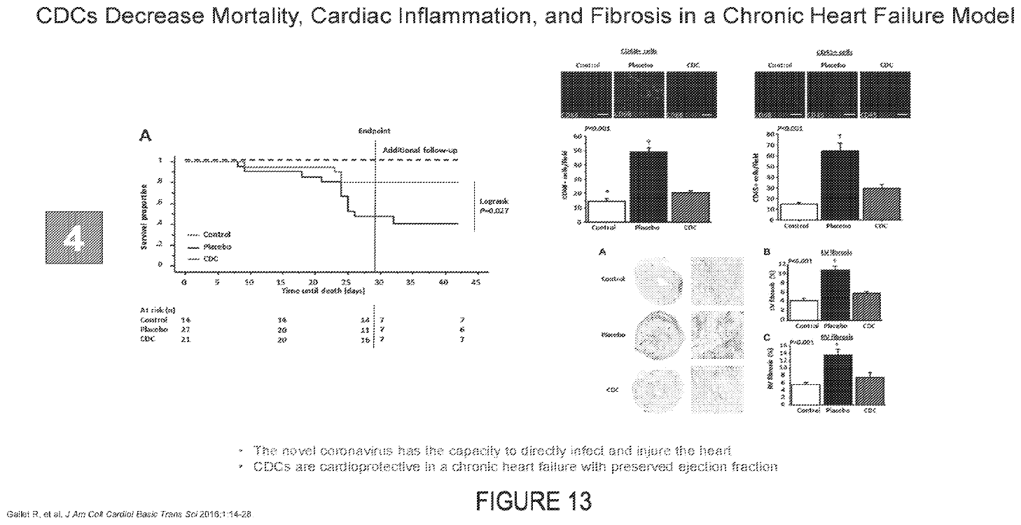

FIG. 13 shows data related to the ability of CDCs to reduce mortality, reduce cardiac inflammation and reduce fibrosis in a chronic heart failure model. The left panel shows that CDC administration improves mortality as compared to placebo. The right panels show reductions in cardiac inflammation and fribrosis. As COVID-19 is associated with direct damage to the heart, in several embodiments, CDCs aid in reducing the pathogenesis of COVID-19, particularly the damage the SARS-CoV-2 virus can induce directly in the heart.

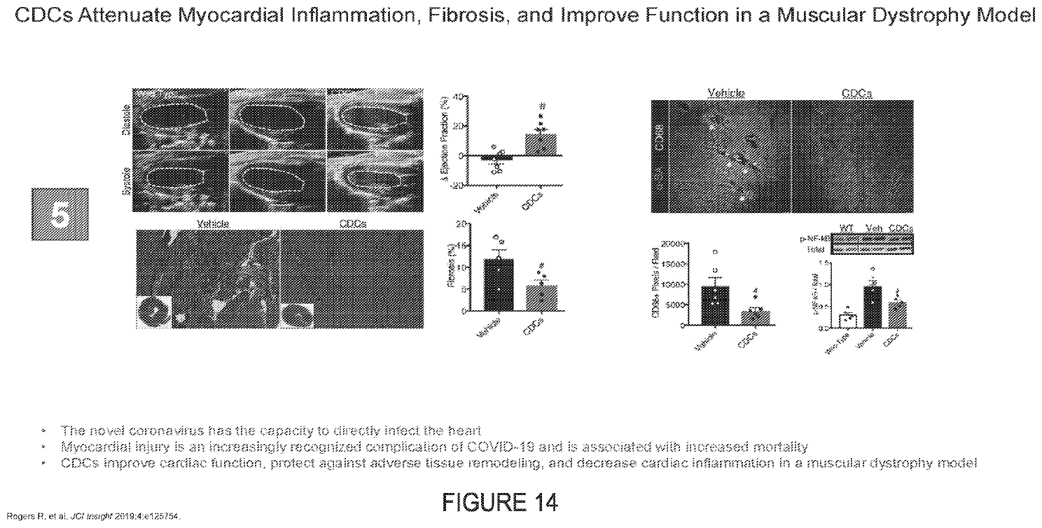

Further supporting the potential for CDC administration to reduce the direct heart damage seen in COVID-19 is shown in FIG. 14. FIG. 14 shows that myocardial inflammation is attenuated after CDC administration, fibrosis is reduced and cardiac function is improved in a muscular dystrophy model. The left panel (upper row) shows echo and summary data of increased ejection fraction seen after CDC administration. The left panel (lower row) shows reduced fibrosis after CDCs. The right panel shows reduced CD68 detection with CDC administration (reduced macrophage infiltration) and reduced NF-Kb, a marker of cardiac tissue remodeling.

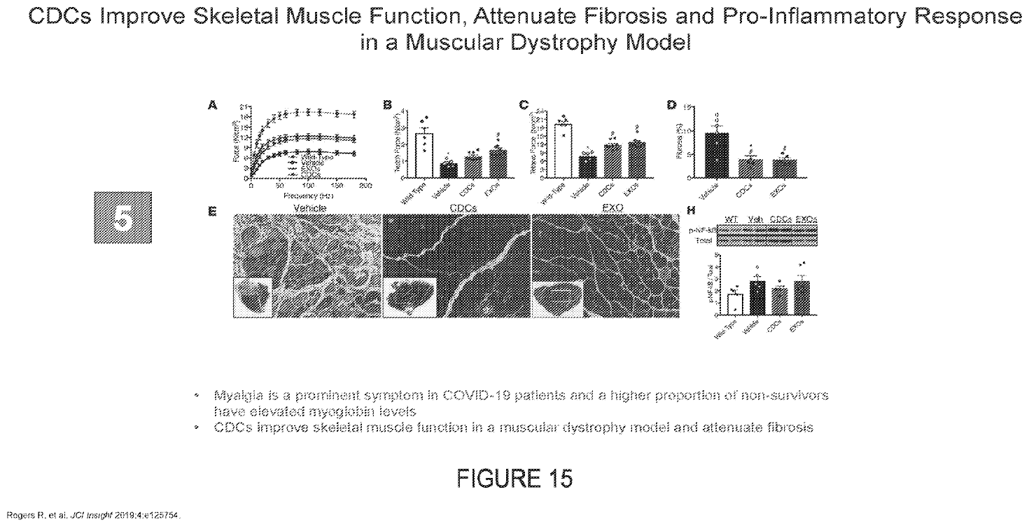

FIG. 15 shows that skeletal muscle function is improved in a muscular dystrophy model after CDC administration or CDC-derived exosomes. (see FIG. 15 top left three panels). FIG. 15, panel D, shows recued fibrosis with CDCs and CDC-derived exosomes.the lower panels show that CDCs or CDC-derived exosomes reduce fibrosis and NF-kB expression, indicated they reduce adverse tissue remodeling. These effects could be benefical in reducing the adverse effects of COVID-19, one of which is myalgia.

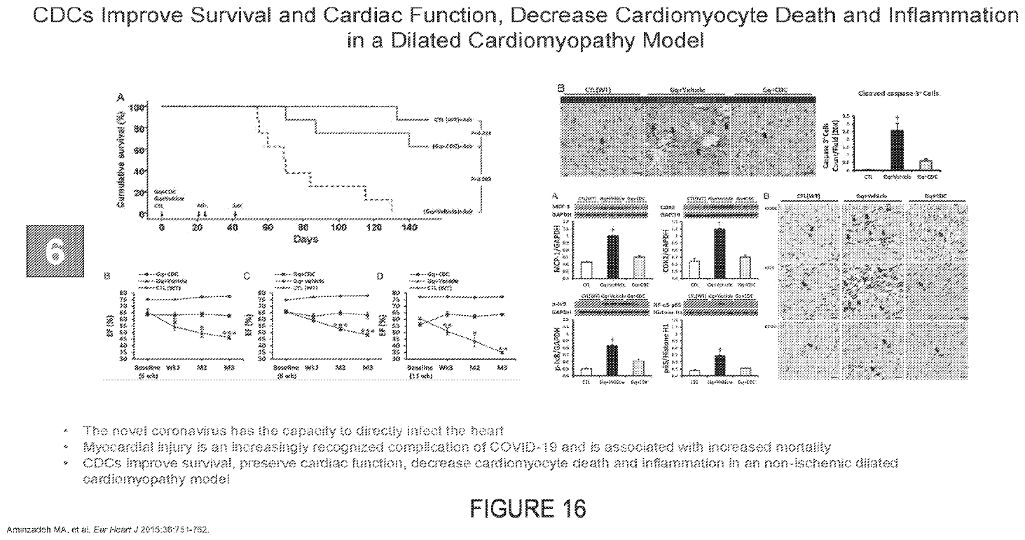

CDCs, as shown in FIG. 16, improve survival and cardiac function, decrease cariomycoyte death and reduce inflammation in dialted cardiomyopathy model. Direct myocardial injury is a symptom of some viral infections, like COVID-19. Thus, the preservation of function and reduction in cardiomyocyte death and inflammation are beneficial, in several embodiments, in reducing cardiac injury in COVID-19.

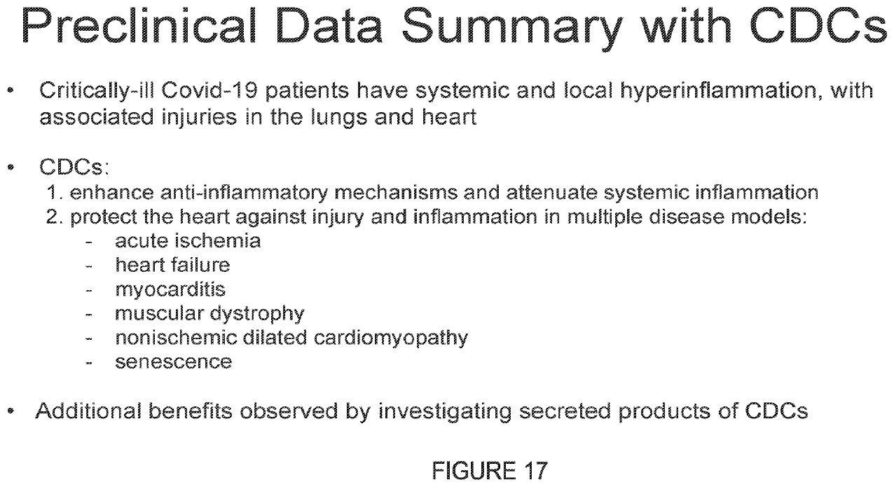

FIG. 17 provides a summary of the potential positive effects and putative mechanisms of action of CDCs on improving outcomes for COVID-19 patients. As discussed, critically-ill Covid-19 patients have systemic and local hyperinflammation, with associated injuries in the lungs and heart. Beneficially, CDC administration has been shown to enhance anti-inflammatory mechanisms and attenuate systemic inflammation as well as protect the heart against injury and inflammation in multiple disease models (including acute ischemia, heart failure, myocarditis, muscular dystrophy, nonischemic dilated cardiomyopathy, and senescence). Additional benefits have also been observed by investigating secreted products of CDCs.

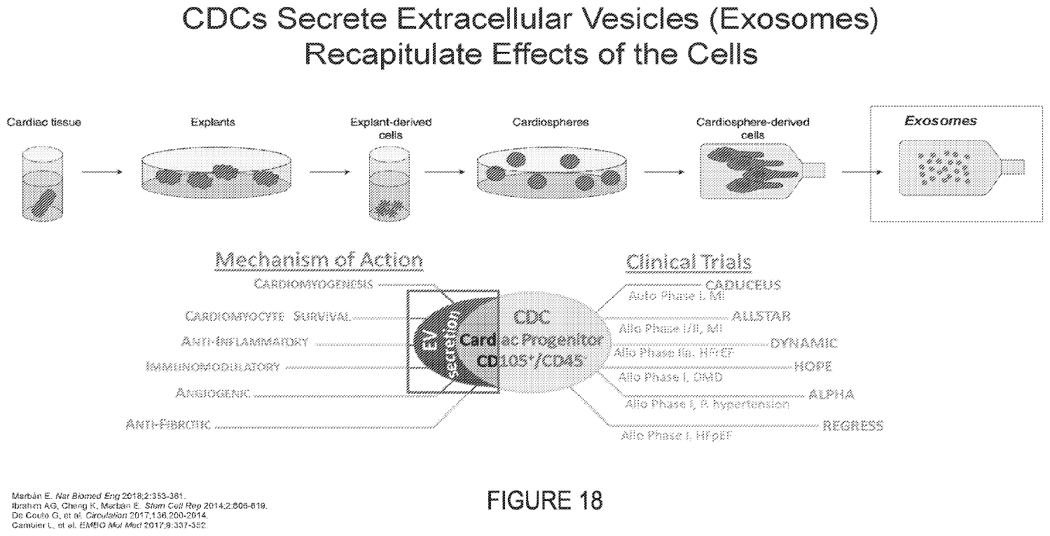

As discussed above, in several embodiments, CDC-derived exosomes are used for the treatment of viral infection, such as COVID-19. FIG. 18 shows a schematic of the production process for CDC-derived exosomes (discussed in more detail above). FIG. 19 shows a schematic of the potential therapeutic targets for CDC-derived exosomes in the pathogenesis of COVID-19 (though these targets may also apply to other viral infections). As with CDCs, CDC derived exosomes show the ability to enhance cell debris clearance, descrease proinflammatory cytokine production, increase anti-inflammatory cytokine production, increase proliferation of anti-inflammatory Tregs as well as a host of additional salutary effects. As with the CDCs, the data discussed for CDC-derived exosomes is tied to the pathogenic effect by the large number that corresponds to the numbers on the schematic of FIG. 19.

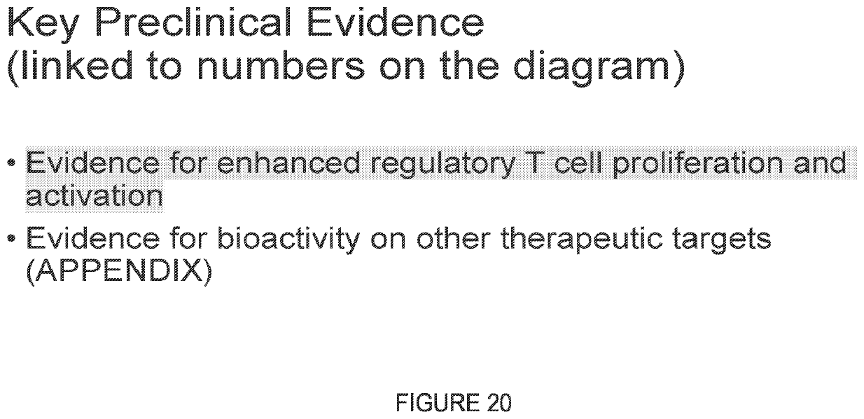

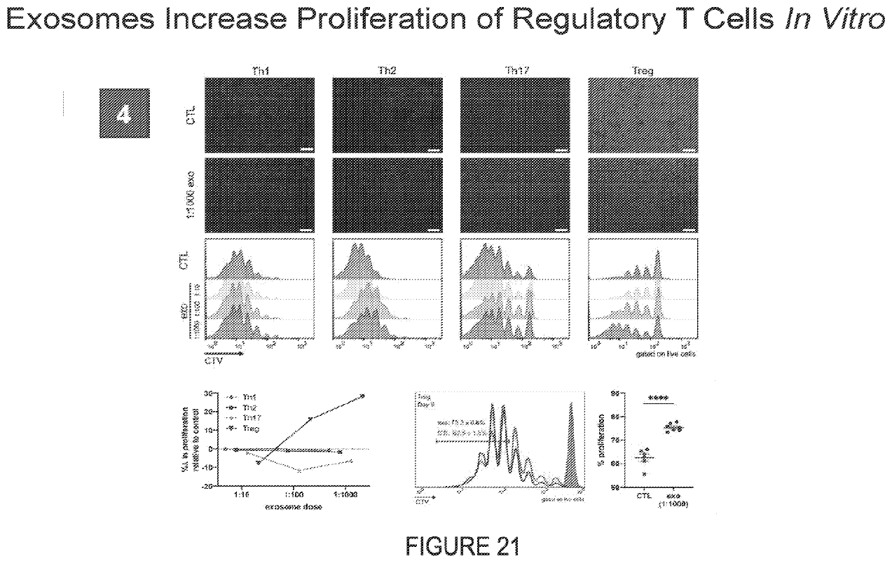

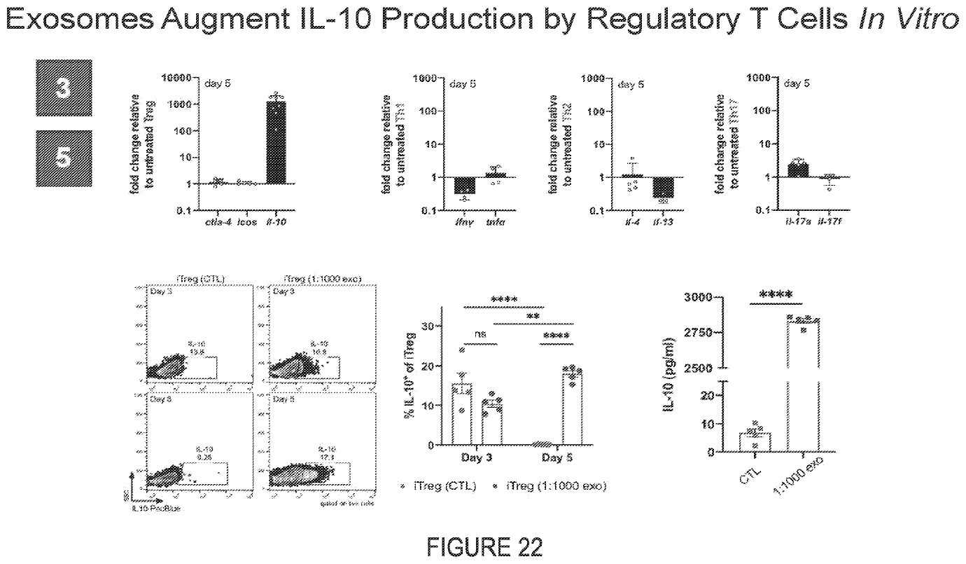

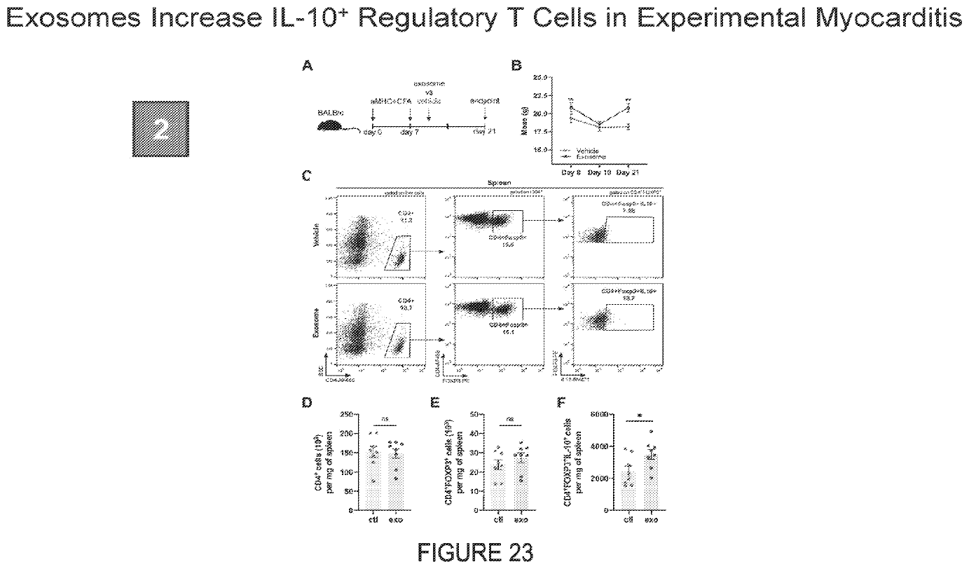

FIG. 20 provides an explanation of the content of FIGS. 21-35. FIG. 21 shows that exosomes have the ability to induce proliferation of regulatory T cells in vitro. FIG. 22 provides data that evidences that exosomes augment the production of IL10 by regulatory T cells. The flow cytometry data in the lower left show that IL10 secretion is increased as a result of co-culture of Treg cells with CDC-derived exosomes. The lower right panels show further data related to increased IL10 secretion by Treg cells due to exposure to CDC-derived exosomes. FIG. 23 shows that exosomes increase IL10 positive Treg cells in an experimental myocarditis model.

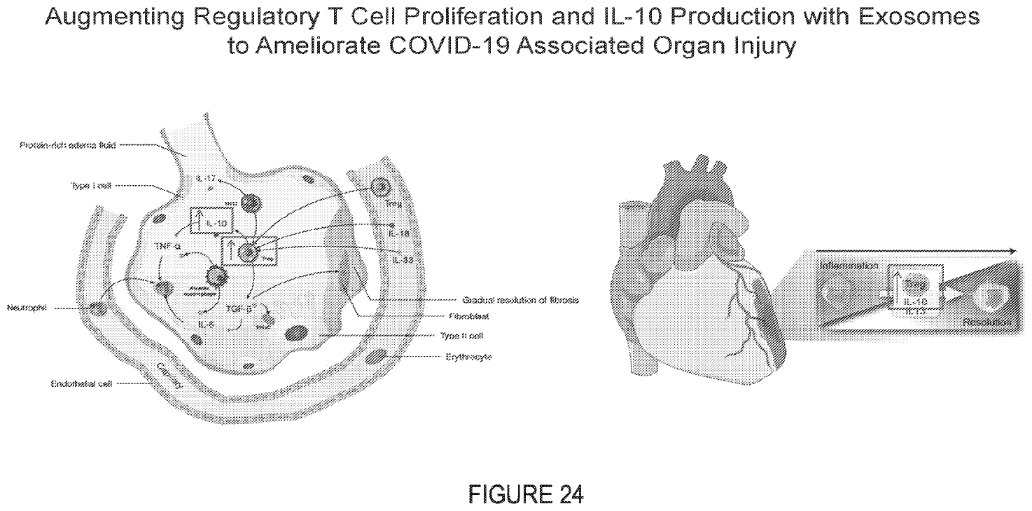

FIG. 24 shows a schematic as to how the increase of IL10 production and Treg production can gradually reduce fibrosis in the lungs (left) and the heart (right).

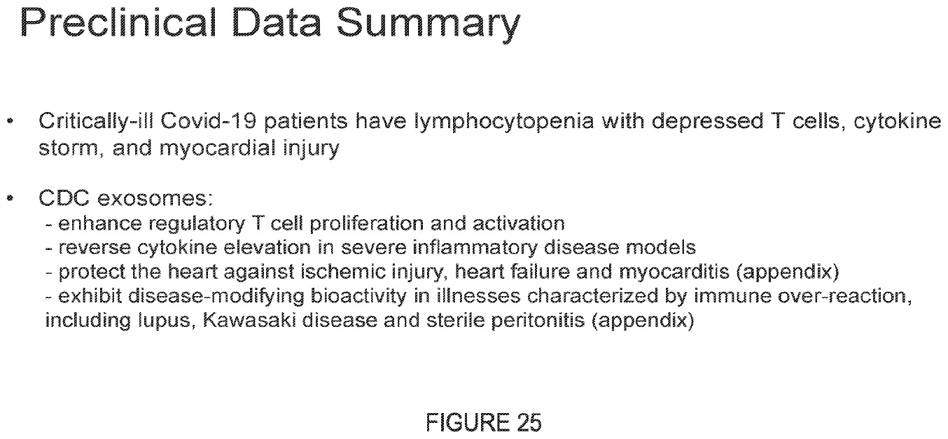

FIG. 25 shows a summary of the preclinical data shown in FIGS. 18-24. Critically-ill Covid-19 patients have lymphocytopenia with depressed T cells, cytokine storm, and myocardial injury. CDC-derived exosomes can help reduce the pathogenesis of viral infections, such as COVID-19, as a result of the CDC-derived exosomes to enhance regulatory T cell proliferation and activation, reverse cytokine elevation in severe inflammatory disease models, protect the heart against ischemic injury, heart failure and myocarditis, and/or exhibit disease-modifying bioactivity in illnesses characterized by immune over-reaction, including lupus, Kawasaki disease and sterile peritonitis (all discussed in brief below).

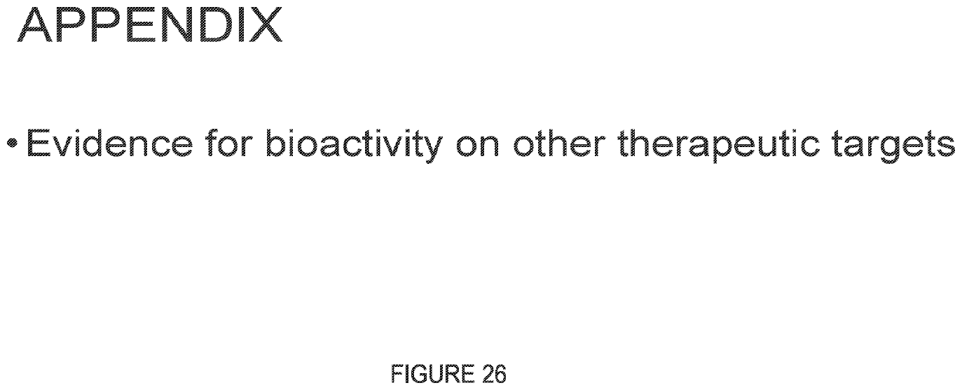

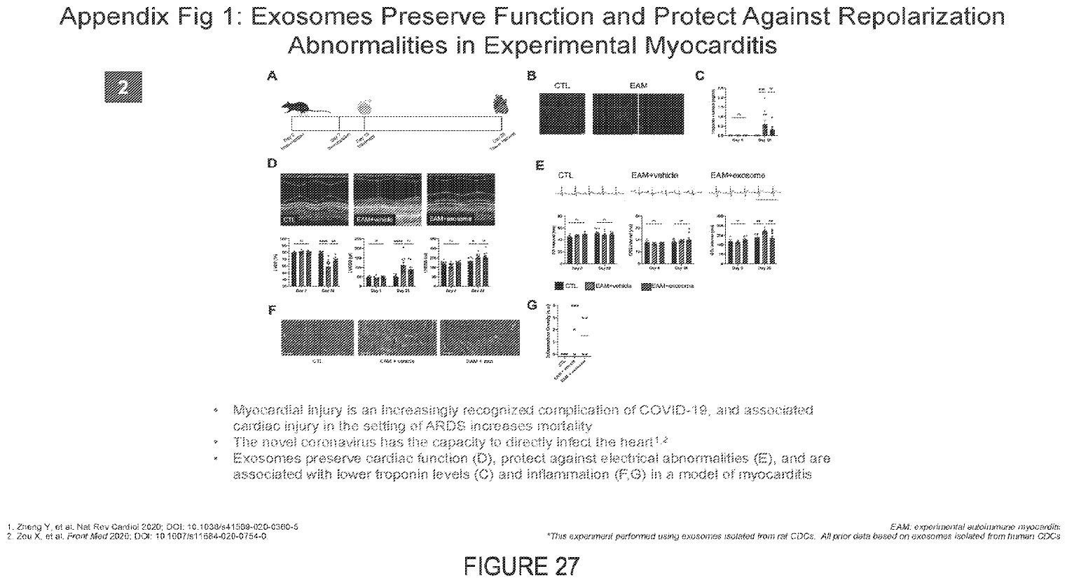

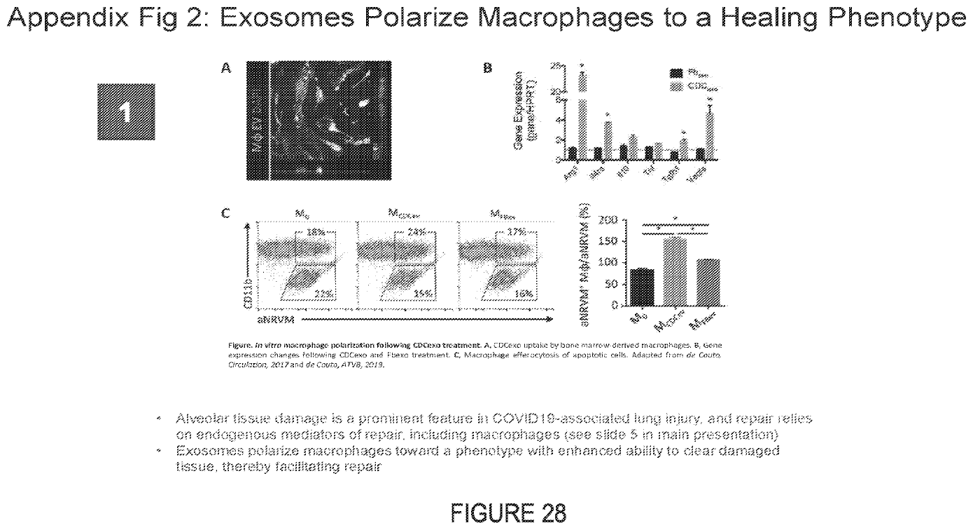

FIG. 26 provides an explanation of the content of FIGS. 27-35. As discussed above, myocardial injury is an increasingly recognized complication of COVID-19, and associated cardiac injury in the setting of ARDS increases mortality. SARS-CoV-2 has the capacity to directly infect the heart. However, to combat this injury of the cardiac tissue, the data shown in FIG. 27 demonstrate that CDC-derived exosomes preserve cardiac function (D), protect against electrical abnormalities (E), and are associated with lower troponin levels (C) and inflammation (F,G) in a model of myocarditis.

Additionally, alveolar tissue damage is a prominent feature in COVID-19-associated lung injury, and repair relies on endogenous mediators of repair, including macrophages (see FIG. 5). However, as discussed above with respect to CDCs, and shown in FIG. 28, CDC-derived exosomes polarize macrophages toward a phenotype with enhanced ability to clear damaged tissue, thereby facilitating repair. In several embodiments, this allows exosomes to increase the efferocytosis of apoptotic cells which can limit further damage to lung or cardiac tissue and assist in facilitating repair.

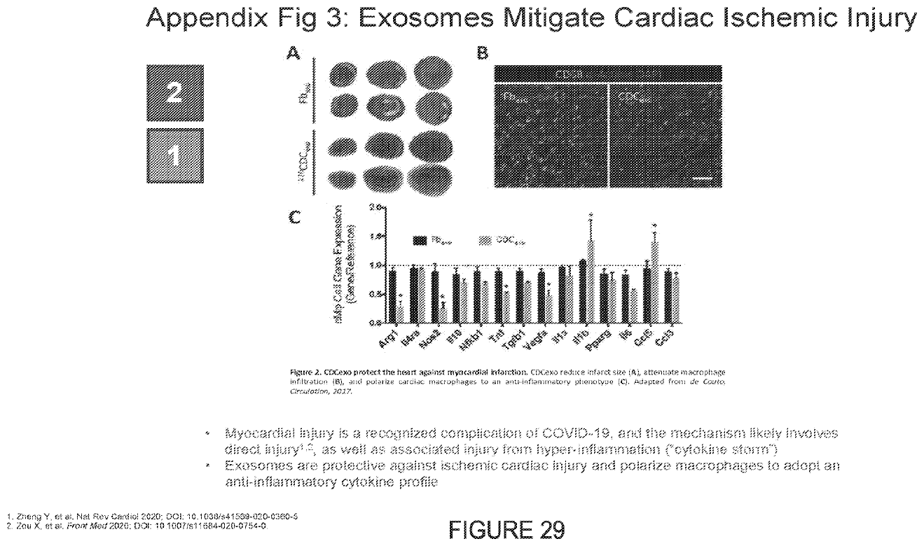

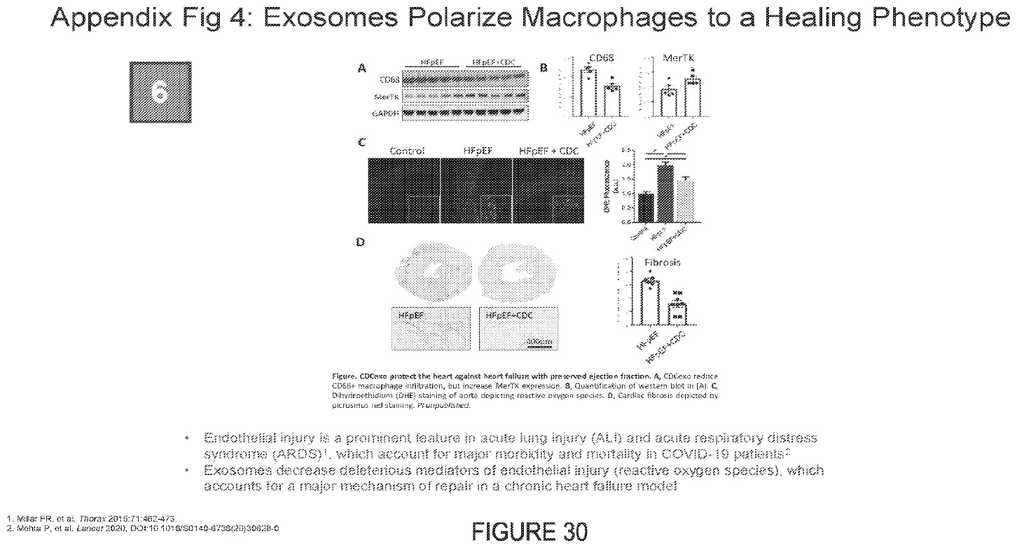

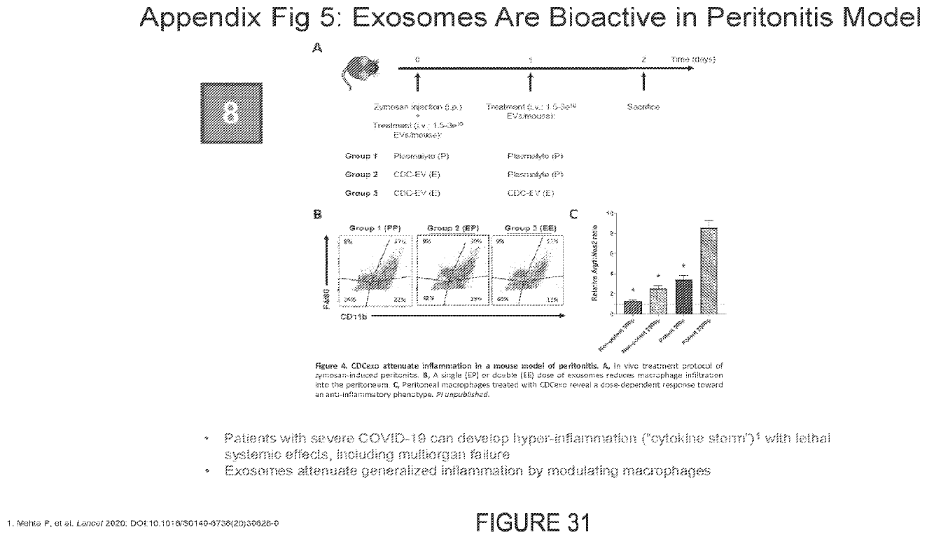

FIG. 29 relates to the ability of CDC-derived exosomes to mitigate cardiac ischemic injury. COVID-19 is associated with direct myocardial injury, and the mechanism is purported to be related to the cytokine storm/hyperinflammation. Exosomes, however, are protective against ischemic cardiac injury, FIG. 29B shows reduced macrophage infiltration in CDC-derived exosome-treated hearts. FIG. 29A shows reduction in infarct size by CDC-derived exosome treatment. The lower panel (FIG. 29C) shows gene expression profiles for macrophages, with macrophages from animals treated with CDC-derived exosomes expression of the indicated cytokines, which demonstrate an anti-inflammatory phenotype. Further data is shown in FIG. 30, which relates to the CDC-derived exosomes ability to decrease deleterious mediators of endothelial injury (reactive oxygen species), which accounts for a major mechanism of repair in a chronic heart failure model. In the context of a viral infection, endothelial injury is a prominent feature in acute lung injury (ALI) and acute respiratory distress syndrome (ARDS), which account for major morbidity and mortality, such as in COVID-19 patients. Thus the ability of CDC-derived exosomes to reduce underlying mediator of such symptoms, in several embodiments, aids in the treatment of viral infections, such as COVID-19.

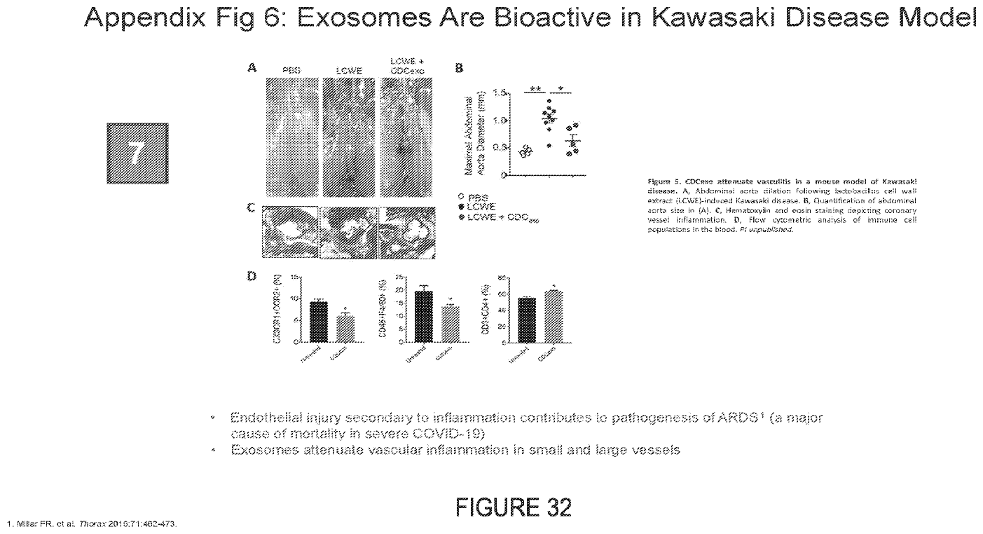

Further supportive data relating to CDC-derived exosomes to address symptoms of viral infection, such as COVID-19, is shown in FIG. 31. These data show that exosomes are biologically active in a model of peritonitis. FIG. 31A is a schematic diagram of an experimental design for the peritonitis model. FIG. 31B shows that single (EP) or double (EE) dose of exosomes reduces macrophage infiltration into the peritoneum. FIG. 31C shows that peritoneal macrophages treated with CDC-derived exosomes (CDCexo) reveal a dose-dependent response toward an anti-inflammatory phenotype. Thus, CDC-derived exosomes, in several embodiments, aid in the treatment of viral infections, such as COVID-19.

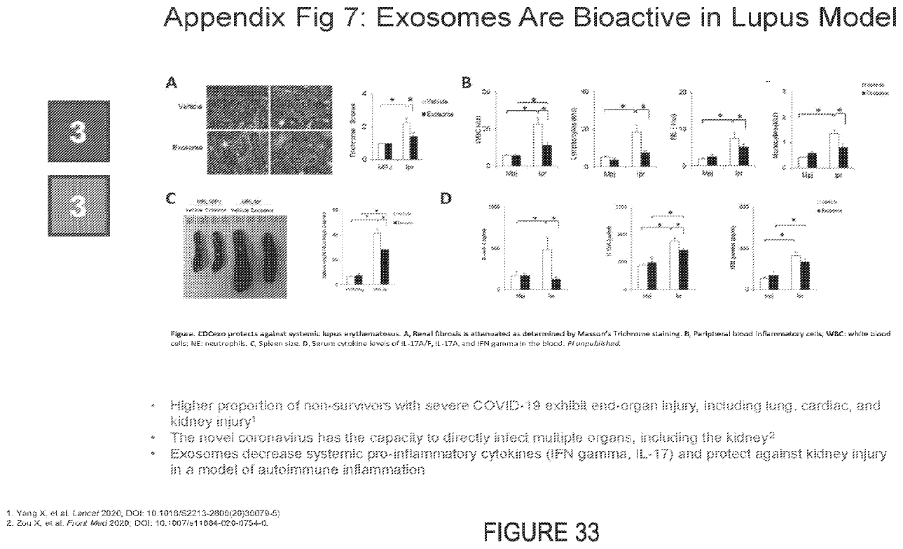

FIG. 32 shows additional supportive data. As discussed above, endothelial injury secondary to inflammation contributes to pathogenesis of ARDS (a major cause of mortality in severe COVID-19). However, the data in FIG. 32 show that CDC-derived exosomes attenuate vascular inflammation in small and large vessels (using a Kawasaki Disease model). Notably, maximal abdominal aorta diameter is reduced in CDC-derived exosome treated animals, indicative of the exosomes ability to reduce inflammation, which could be beneifical in trating viral infections, such as COVID-19.

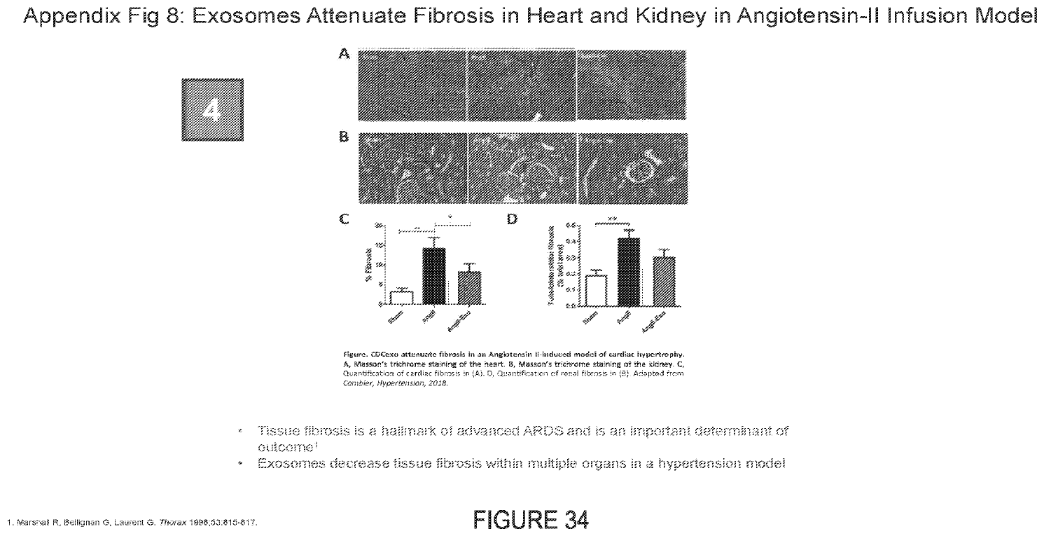

FIG. 33 shows data related to the efficacy of CDC-derived exosomes in a Lupus Model, where they decrease systemic pro-inflammatory cytokines (IFN gamma, IL-17) and protect against kidney injury. These data suggest that the CDC-derived exosomes could be beneficial in the reduction of viral-induced damage to lung, cardiac and/or kidney tissues.

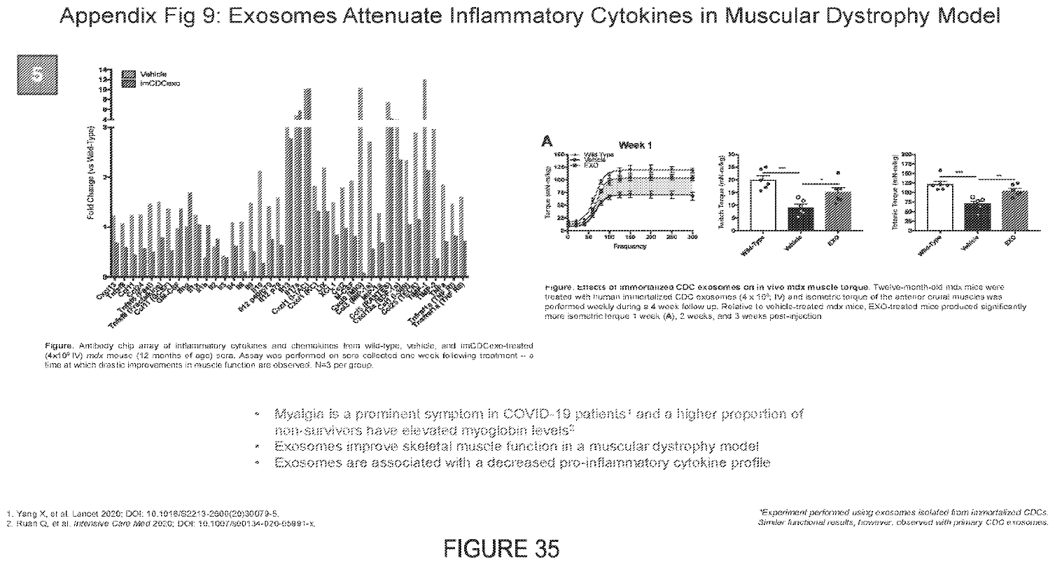

FIG. 34 shows data related to the attenuation of fibrosis in the heart and the kidney in an Angiotensin-II infusion model. Tissue fibrosis is a hallmark of advanced ARDS, a symptom of COVID-19, and is an important determinant of outcome. Thus, the ability of CDC-derived exosomes to decrease tissue fibrosis within multiple organs in a hypertension model suggests that, as according to several embodiments herein, CDC-derived exosomes are beneficial in reducing the fibrosis associated with COVID-19, or other viral infections.

FIG. 35 relates to the ability of CDC-derived exosomes to attenuate inflammatory cytokine release in a muscular dystrophy model. Myalgia is a prominent symptom in COVID-19 patients and a higher proportion of non-survivors have elevated myoglobin levels. Thus, the ability of CDC-derived exosomes to improve skeletal muscle function in a muscular dystrophy model and to induce a decreased pro-inflammatory cytokine profile suggests that, as according to several embodiments herein, CDC-derived exosomes are beneficial in reducing the fibrosis associated with COVID-19, or other viral infections.

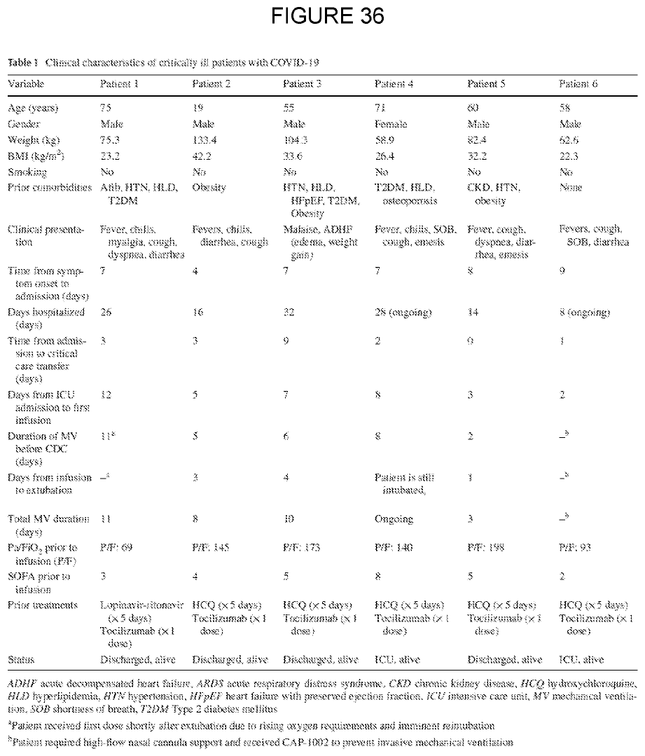

FIG. 36 is a table listing clinical characteristics of COVID-19 patients treated with CDCs, according to some embodiments of the present disclosure.

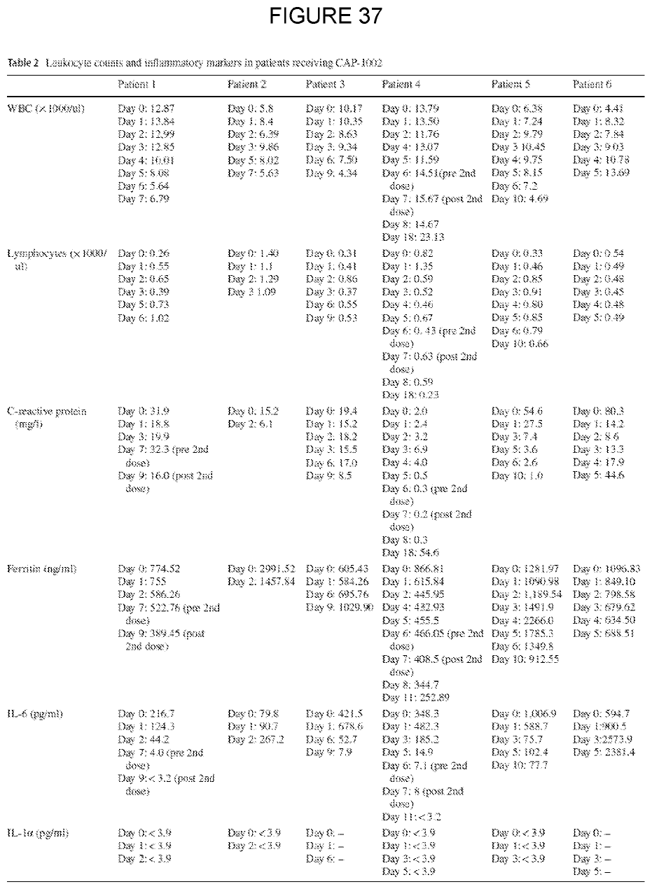

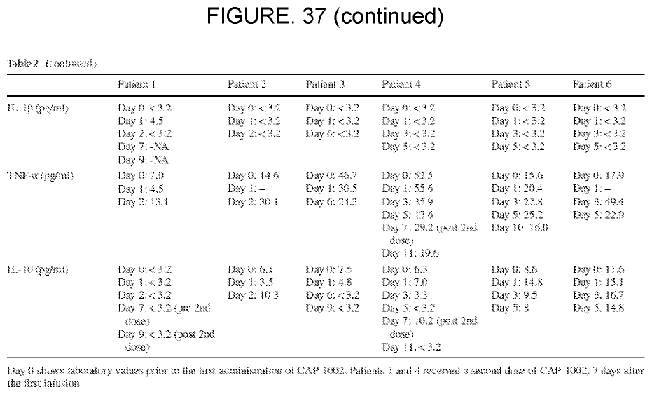

FIG. 37 is a table summarizing blood cell counts, inflammatory markers and cytokine levels in COVID-19 patients treated with CDCs, according to some embodiments of the present disclosure.

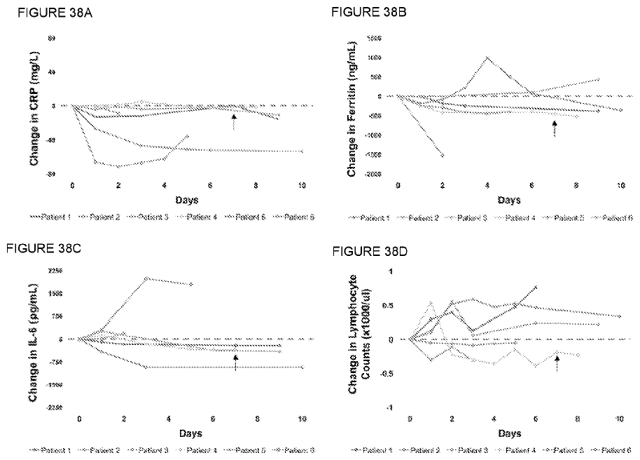

FIGS. 38A-38D are a collection of graphs showing trends in inflammatory markers and lymphocyte counts. Changes in the level of inflammatory markers, such as C-reactive protein (CRP) (FIG. 38A), ferritin (FIG. 38B), and IL-6 (FIG. 38C), and lymphocyte counts (FIG. 38D), normalized to baseline values (preinfusion), within 10 days of CAP-1002 infusion in COVID-19 patients, according to some embodiments of the present disclosure. Dashed arrows indicated the time of second infusion.

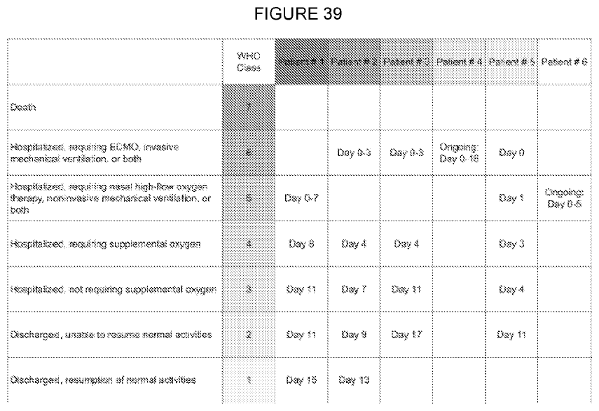

FIG. 39 is a schematic diagram summarizing the clinical course of individual COVID-19 patients treated with CDCs, according to some embodiments of the present disclosure.

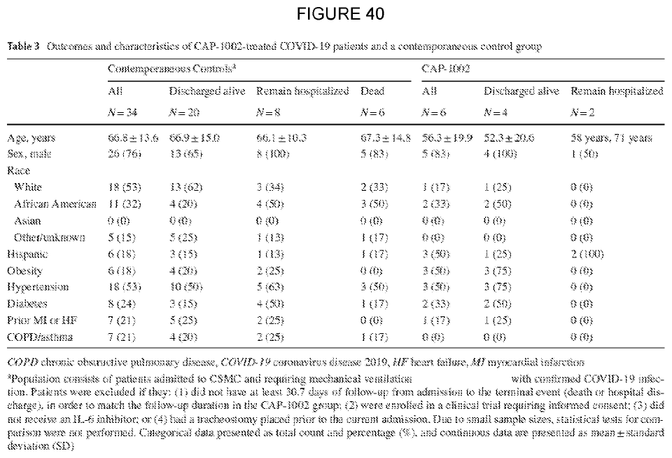

FIG. 40 is a table comparing clinical outcome and characteristics of COVID-19 patients treated with CDCs with a contemporaneous control group, according to some embodiments of the present disclosure.

### METHODS OF TREATMENT, ADMINISTRATION AND DOSING

Some embodiments relate to a method of treating, ameliorating, inhibiting, or preventing viral infection through the administration of CDCs, CDC-derived exosomes, and/or concomitant CDC/CDC-derived exosome administration with an additional therapeutic agent.

In some embodiments, a method of treating a viral infection includes administering to a subject in need of treating a viral infection, e.g., a respiratory viral infection, a therapeutically effective amount of cardiosphere-derived cells (CDCs) and/or CDC-derived exosomes, thereby treating the viral infection and/or one or more symptoms or sequelae thereof. In some embodiments, the method includes administering a therapeutically effective amount of CDCs to the subject. In some embodiments, the method includes administering a therapeutically effective amount of CDC-derived exosomes to the subject. In some embodiments, the respiratory viral infection is COVID-19 or variant thereof.

In some embodiments, administering the therapeutically effective amount of CDCs and/or CDC-derived exosomes reduces a hyperinflammatory response, or sequelae thereof, associated with a SARS-CoV-2 infection. In some embodiments, administering the therapeutically effective amount of CDCs and/or CDC-derived exosomes reduces one or more of respiratory distress, myocardial injury, and/or inflammatory cytokine level. In some embodiments, administering the therapeutically effective amount of CDCs and/or CDC-derived exosomes reduces, ameliorates or eliminates respiratory distress. In some embodiments, administering the therapeutically effective amount of CDCs and/or CDC-derived exosomes reduces, repairs or restores myocardial injury. In some embodiments, administering the therapeutically effective amount of CDCs and/or CDC-derived exosomes reduces the subject's inflammatory cytokine level, including, without limitation, the level of one or more of IL-6, TNF (tumor necrosis factor) α, IL-2, IL-7, IFN (interferon)-γ IP (inducible protein)-10, MCP (monocyte chemoattractant protein)-1, MIP (macrophage inflammatory protein)-1α, and G-CSF (granulocyte-colony stimulating factor). In some embodiments, the inflammatory cytokine level is reduced by about 5%, 10%, 15%, 20%, 25%, 30%, 40%, 50%, 60%, 70%, 80%, 90%, 95%, 100%, or by a percentage in a range between any two of the preceding values, after administering the therapeutically effective amount of CDCs and/or CDC-derived exosomes.

In some embodiments, a method of treating a viral infection includes administering to a subject in need of treating a viral infection, e.g., a respiratory viral infection, a population of cardiosphere-derived cells (CDCs) and/or CDC-derived exosomes, wherein the population comprises about 1×10⁸ to about 2×10⁸ CDCs and/or about 2×10⁸ to about 2×10¹⁰ CDC-derived exosomes; and optionally administering to the subject at least one additional therapeutic, wherein the additional therapeutic is selected from an antihistamine, anti-inflammatory agent, immunosuppressive agent, cyclooxygenase-2 inhibitor, TNF-α inhibitor, cytokine inhibitor or a combination thereof; wherein administering the CDCs and/or CDC-derived exosomes and the at least one additional therapeutic reduce one or more of respiratory distress, myocardial injury, and/or inflammatory cytokine level, thereby treating the viral infection. In some embodiments, the respiratory viral infection is COVID-19 or variant thereof.

In certain embodiments, treatment of a subject with a CDCs, CDC-derived exosomes, and/or an additional therapeutic agent as described herein achieves one, two, three, four, or more of the following effects, including, for example: (i) reduction or amelioration in the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. As used herein, treatment of a subject with CDCs and/or CDC-derived exosomes, e.g., for a viral infection, such as a respiratory viral infection, or a symptom thereof contemplates treating one or more sequelae or complications of a disease, e.g., viral infection, or symptom thereof. In some embodiments, methods of the present disclosure find use in preventing, reducing the likelihood of, or reducing the severity of one or more sequelae or complications of the disease, e.g., viral infection, or symptom thereof. In some embodiments, methods of the present disclosure find use in treating or ameliorating one or more sequelae or complications of a viral infection or symptom thereof after the subject is free of the viral infection. A variety of sequelae or complications of a viral infection, e.g., COVID-19, or symptom thereof can be treated by the present methods. In some embodiments, sequelae or complications of the viral infection, e.g., COVID-19, or symptom thereof include, without limitation, pulmonary damage and cardiac injury. In some embodiments, administration of a therapeutically effective amount of cardiosphere-derived cells (CDCs) and/or CDC-derived exosomes to the subject achieves the above results.

The term “effective amount” as used herein refers to the amount of a composition or an agent sufficient to provide the desired effect, e.g., cellular response, therapeutic effect, etc. The term “therapeutically effective amount” refers to an amount of a therapeutic composition or therapeutic agent that is sufficient to provide a beneficial (e.g., clinical or otherwise) effect when administered to a typical subject. A therapeutically effective amount as used herein, in various contexts, can include an amount sufficient to delay the development of a disease, or symptom or sequelae thereof, alter the course of a disease, or symptom or sequelae thereof (for example but not limited to, slowing the progression of a disease, or symptom or sequelae thereof), or reverse a symptom of the disease. The therapeutically effective amount may be administered in one or more doses of the therapeutic agent. The therapeutically effective amount may be administered in a single administration, or over a period of time in a plurality of doses.

Administration can be by a variety of routes, including, without limitation, intravenous, intra-arterial, subcutaneous, intramuscular, intrahepatic, intraperitoneal and/or local delivery to an affected tissue.

The subject treated by the present methods can be any suitable subject in need of treatment of a viral infection. In some embodiments, the subject is a mammal. In some embodiments, the subject is a human subject. In some embodiments, the subject has one or more comorbidities, such as, but not limited to a cardiorespiratory dysfunction, hypertension, diabetes, and/or coronary heart disease. “Subject” and “patient” are used interchangeably herein.

In some embodiments, the subject in need of treatment has severe COVID-19, e.g., where a more aggressive medical intervention is required. In some embodiments, the subject is on ventilatory support, including, without limitation, on high-flow nasal cannula (HFNC) support, or intubation. In some embodiments, the subject is in an intensive care unit (ICU). In some embodiments, where the subject has severe COVID-19, a method of the present disclosure shortens the duration of the aggressive medical intervention. In some embodiments, a method of the present disclosure reduces length of hospital stay, length of stay in an ICU, or duration of ventilatory support. In some embodimenets, the duration of the aggressive medical intervention is reduced by 1, 2, 3, 4, 5, 6, 7, 8, 9, 10, 11, 12, 13, 14 days or more after administering the CDCs and/or CDC-derived exosomes to the subject, compared to individuals having a similar disease severity but do not receive the CDCs and/or CDC-derived exosomes. In some embodiments, the subject has yet to develop symptoms of severe COVID-19, and a method of the present disclosure prevents development of severe COVID-19, or reduces the likelihood of developing severe COVID-19 compared to individuals who do not receive the CDCs and/or CDC-derived exosomes.

In some embodiments, the subject in need of treatment exhibits cardiac involvement associated with the viral infection, e.g., COVID-19. In some embodiments, the subject has acute myocarditis. In some embodiments, the subject has arrhythmia. In some embodiments, the subject has or has suffered a myocardial infarction.

In some embodiments, the subject in need of treatment has ARDS. In some embodiments, the subject has hypoxemia. In some embodiments, the subject suffers from hyperinflammation or a cytokine storm (e.g., exaggerated systemic inflammation). In some embodiments, the subject has elevated levels of an inflammatory marker or inflammatory cytokine, such as, without limitation, ferritin, CRP (C-reactive protein), IL-6, TNF (tumor necrosis factor) α, IL-2, IL-7, IFN (interferon)-γ IP (inducible protein)-10, MCP (monocyte chemoattractant protein)-1, MIP (macrophage inflammatory protein)-1α, G-CSF (granulocyte-colony stimulating factor), and procalcitonin. In some embodiments, the subject has elevated levels of ferritin, CRP, IL-6, TNFα. In some embodiments, the subject has elevated levels of cardiac Tropnonin I (TnI) and/or BNP (Brain-type natriuretic peptide). The level of an inflammatory marker, inflammatory cytokine or a biomarker can be elevated relative to a suitable control level, e.g., level of the maker or cytokine in a healthy cohort.

In some embodiments, the subject in need of treatment has lymphopenea (or lymphocytopenea). In some embodiments, the subject has a lymphocyte count of about 1000/μL or less, about 900/μL or less, about 800/μL or less, about 700/μL or less, about 600/μL or less, about 500/μL or less, about 400/μL or lesss, about 300/μL or less, about 200/μL or less, or a lymphocyte count within a range defined by any two of the preceding values. In some embodiments, the subject has a progressive lymphopenea, e.g., that progressively decreases or does not improve over time, before administering the CDCs or CDC-derived exosomes. In some embodiments, the subject's lymphocyte count recovers or does not decrease further over time after administering a therapeutically effective amount of CDCs or CDC-derived exosomes to the subject.

In some embodiments, the CDCs and/or CDC-derived exosomes are administered in conjunction with one or more therapies to treat the disorder or disease. In some embodiments, the CDCs and/or CDC-derived exosomes are administered after, before, or concurrently with administration of one or more therapeutic agents.

The therapeutic agent can be any suitable therapeutic agent for treating the disorder or disease, or one or more symptoms thereof, such as, but not limited to: analgesic agents; anti-arthritic agents; anti-asthmatic agents; anticancer agents; anticholinergic agents; antihistamines; anti-infective agents; anti-inflammatory agents; cardiovascular medicaments; gastrointestinal medicaments; immunosuppressive agents; leukotriene inhibitors; narcotic agonists and antagonists; peptide drugs; phytonutrients; vasodilators; vitamins and mineral supplement; and combinations thereof.

In some embodiments, the therapeutic agent is an anti-inflammatory agent. In some embodiments, the anti-inflammatory agent is a corticosteroid, such as, without limitation, cortisone hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, methylprednisolone, betamethasone, typically as betamethasone benzoate or betamethasone diproprionate; fluocinonide; prednisone; and triamcinolone, typically as triamcinolone acetonide. In some embodiments, the anti-inflammatory agent is a mast cell degranulation inhibitor, such as, without limitation, cromolyn (5,5′-(2-hydroxypropane-1,3-diyl)bis(oxy)bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-dicarboxylic acid) and tranilast (2-{[(2E)-3-(3,4-dimethoxyphenyl)prop-2-enoyl]amino}), and lodoxamide (2-[2-chloro-5-cyano-3-(oxaloamino)anilino]-2-oxoacetic acid). In some embodiments, the anti-inflammatory agent is a nonsteroidal anti-inflammatory drugs (NSAIDs), such as, without limitation, aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin.

In some embodiments, the anti-inflammatory agent is an antihistamine, such as, without limitation, clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine and promethazine, and diphenhydramine, or pharmaceutically acceptable salts, solvates or esters thereof. In some embodiments, the antihistamine includes, without limitation, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. In some embodiments, the antihistamine is an H1 antagonist, such as, but not limited to, cetirizine, chlorpheniramine, dimenhydrinate, diphenhydramine, fexofenadine, hydroxyzine, orphenadrine, pheniramine, and doxylamine. In some embodiments, the antihistamine is an H2 antagonist, such as, but not limited to, cimetidine, famotidine, lafutidine, nizatidine, ranitidine, and roxatidine.

In some embodiments, the therapeutic agent is an antiviral agent, including antiretroviral agents. Suitable antiviral agents include, without limitation, remdesivir, acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, ribavirin and rimantadine.

In some embodiments, the therapeutic agent is an antibiotic. Non-limiting examples of suitable antibiotics include beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; Carbapenems such as imipenem and eropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tlimicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadmethoxine, sfulfamethazine, sulfaquinoxaline, sulfamerazine, sulfathiazole, sulfasalazine, sulfadiazine, sulfabromomethazine, suflaethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.).

In some embodiments, the therapeutic agent is an antifungal agent, such as, but not limited to, itraconazole, ketoconazole, fluoconazole, and amphotericin B. In some embodiments, the therapeutic agent is an antiparasitic agents, such as, but not limited to, the broad spectrum antiparasitic medicament nitazoxanide; antimalarial drugs and other antiprotozoal agents (e.g., artemisins, mefloquine, lumefantrine, tinidazole, and miltefosine); anthelminthics such as mebendazole, thiabendazole, and ivermectin; and antiamoebic agents such as rifampin and amphotericin B.

In some embodiments, the therapeutic agent is an analgesic agent, including, without limitation, opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol; and nonopioid analgesics such as apazone, etodolac, diphenpyramide, indomethacin, meclofenamate, mefenamic acid, oxaprozin, phenylbutazone, piroxicam, and tolmetin.

The methods of the present disclosure can provide therapeutic relief to a subject or patient suffering from an infection by one or more of a variety of pathogens. Pathogens include, without limitation, one or more of the following: viruses (including but not limited to coronavirus, human immunodeficiency virus, herpes simplex virus, papilloma virus, parainfluenza virus, influenza virus, hepatitis virus, Coxsackie Virus, herpes zoster virus, measles virus, mumps virus, rubella, rabies virus, hemorrhagic viral fevers, H1N1, and the like), prions, parasites, fungi, mold, yeast and bacteria (both gram-positive and gram-negative). In some embodiments, pathogens include, without limitation, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, and a variety of drug resistant bacteria. In some embodiments, the pathogen is a virus, such as, without limitation, a respiratory virus. In some embodiments, respiratory virus is, without limitation, an influenza virus, respiratory syncytial virus, parainfluenza virus, metapneumovirus, rhinovirus, coronavirus, adenovirus, or bocavirus.

In some embodiments, the pathogen is a virus, e.g., a DNA or RNA virus. In some embodiments, the virus is an RNA virus, e.g., a single or double-stranded virus. In some embodiments, the RNA virus is a positive sense, single-stranded RNA virus. In some embodiments, the virus belongs to the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-CoV), SARS-CoV-2 (COVID-19), Middle Eastern Respiratory Syndrome Coronavirus (MERS-CoV), human coronavirus HKU1, or human coronavirus OC43. In some embodiments, the gammacoronavirus is infectious bronchitis virus (IBV).

### Administration

The CDCs and/or CDC-derived exosomes disclosed herein can be administered by one of many routes, depending on the embodiment. For example, CDC and/or CDC-derived exosome administration may be by local or systemic administration. Local administration, depending on the tissue to be treated, may in some embodiments be achieved by direct administration to a tissue (e.g., direct injection, such as intramyocardial injection). Local administration may also be achieved by, for example, lavage of a particular tissue (e.g., intra-intestinal or peritoneal lavage).

In some embodiments, non-limiting examples of a method to administer a therapeutically effective amount of CDCs and/or CDC-derived exosomes include systemic administration (e.g., intravenous, intra-arterial, intraventricular, intra-aortic, and/or intraperitoneal injection and/or infusion). In some embodiments, the CDCs and/or CDC-derived exosomes are injected or infused intravenously.

In several embodiments, CDC-derived exosomes are specifically targeted to the damaged or diseased tissues. In some such embodiments, the CDC-derived exosomes are modified (e.g., genetically or otherwise) to direct them to a specific target site. For example, modification may, in some embodiments, comprise inducing expression of a specific cell-surface marker on the exosome, which results in specific interaction with a receptor on a desired target tissue. In one embodiment, the native contents of the exosome are removed and replaced with desired exogenous proteins or nucleic acids. In one embodiment, the native contents of exosomes are supplemented with desired exogenous proteins or nucleic acids. In some embodiments, however, targeting of the exosomes is not performed. In several embodiments, exosomes are modified to express specific nucleic acids or proteins, which can be used, among other things, for targeting, purification, tracking, etc. In several embodiments, however, modification of the exosomes is not performed. In some embodiments, the exosomes do not comprise chimeric molecules.

The amount of CDCs and/or CDC-derived exosomes administered to a subject in one or more doses (e.g., in one or more injections or infusions) may vary, and in some embodiments is in a range of about 1×10⁶ to about 1×10¹⁰ per dose, e.g., about 5×10⁶ to about 5×10⁹ per dose, about 2×10⁷ to about 2×10⁹ per dose, about 2×10⁷ to about 1×10⁹ per dose, about 5×10⁷ to about 1×10⁹ per dose, about 5×10⁷ to about 5×10⁸ per dose, about 7×10⁷ to about 5×10⁸ per dose, about 1×10⁸ to about 5×10⁸ per dose, including about 1×10⁸ to about 3×10⁸ per dose, or any dose in between those listed. In some embodiments, the amount of CDCs and/or CDC-derived exosomes administered to a subject in one or more doses (e.g., in one or more injections or infusions) is about 1×10⁶, about 5×10⁶, about 1×10⁷, about 2×10⁷, about 5×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹, about 9×10⁹, about 1×10¹⁰, or about 2×10¹⁰ per dose, or any dose in between those listed. In certain embodiments, the CDC and/or CDC-derived exosome dose is administered on a per kilogram basis, for example, about 1×10⁶/kg to about 1×10¹¹/kg, e.g., about 5×10⁶/kg to about 1×10¹¹/kg, about 5×10⁶/kg to about 5×10¹⁰/kg, about 1×10⁷/kg to about 5×10¹⁰/kg, including about 1×10⁶/kg to about 1×10¹⁰/kg, or any dose in between those listed. In certain embodiments, the amount of CDCs and/or CDC-derived exosomes administered to a subject in one or more doses (e.g., in one or more injections or infusions) is about 1×10⁶/kg, about 2×10⁶/kg, about 5×10⁶/kg, about 1×10⁷/kg, about 2×10⁷/kg, about 5×10⁷/kg, about 1×10⁸/kg, about 2×10⁸/kg, about 5×10⁸/kg, about 1×10⁹/kg, about 2×10⁹/kg, about 5×10⁹/kg, about 1×10¹⁰/kg, about 2×10¹⁰/kg, about 5×10¹⁰/kg, about 1×10¹¹/kg, or any dose in between those listed.

In some embodiments, a therapeutically effective amount of CDCs includes at least about 75×10⁶ to 500×10⁶ CDCs. In some embodiments, a therapeutically effective amount of CDCs includes greater than or equal to about: 75×10⁶ CDCs, 150×10⁶ CDCs, 300×10⁶ CDCs, 400×10⁶ CDCs, 500×10⁶ CDCs, or ranges including and/or spanning the aforementioned values. In some embodiments, a therapeutically effective amount of CDCs includes less than or equal to about: 75×10⁶ CDCs, 150×10⁶ CDCs, 300×10⁶ CDCs, 400×10⁶ CDCs, 500×10⁶ CDCs, or ranges including and/or spanning the aforementioned values.

In some embodiments, the number of CDC-derived exosomes (or CDC-XOs) delivered to the subject in a dose (or dosing regimen) is determined based on the number of CDCs that would be used in a clinically effective dose in a cell-based therapy method. For example, in some embodiments, where 75-500×10⁶ CDCs is an effective dose for therapeutic treatment of skeletal myopathy, using the equivalent amount of CDC-XOs that would be released by those CDCs in vivo would be administered to a patient in a “cell-free” method of treatment. In other words, CDC equivalent doses of CDC-XOs can be used. As an illustration, in some embodiments, 3 mL / 3×10⁸ CDCs, is capable of providing therapeutic benefit. Therefore, a plurality of CDC-XOs as would be derived from that number of CDCs over the time course of those CDCs' residence in the body is used. In some embodiments, the amount of CDC-XOs delivered to the patient is the amount of CDC-XOs that would be released via an injection of equal to or at least about: 75×10⁶ CDCs, 150×10⁶ CDCs, 300×10⁶ CDCs, 400×10⁶ CDCs, 500×10⁶ CDCs, or ranges including and/or spanning the aforementioned values. In some embodiments, the number of CDCs administered in any single dose is 1×10⁵, 1×10⁶, 1×10⁷, 1×10⁸, 1×10⁹, 1×10¹⁰, 1×10¹¹, 1×10¹² (or ranges including and/or spanning the aforementioned values). In some embodiments, the amount of CDC-XOs delivered to the patient is the amount of CDC-XOs that would be released via an injection of equal to or at least about: 1×10⁵ CDCs, 1×10⁶ CDCs, 1×10⁷ CDCs, 1×10⁸ CDCs, 1×10⁹ CDCs, 1×10¹⁰ CDCs, 1×10¹¹ CDCs, 1×10¹² CDCs, or ranges including and/or spanning the aforementioned values. In some embodiments, a dose of CDCs ranges between about 10 and 90 million CDCs, including about 10 to about 20 million, about 20 to about 30 million, about 30 to about 50 million, about 50 to about 60 million, about 60 to about 70 million, about 70 to about 75 million, about 75 million to about 80 million, about 80 million to about 90 million, and ranges including and/or spanning the aforementioned values. Some such does are particularly favorable for coronary delivery. In several embodiments, the dose of CDCs ranges from about 30 million to about 1.5 billion CDCs, including about 30 million to about 45 million, about 40 million to about 50 million, about 50 million to about 50 million, about 60 to about 75 million, about 75 to about 1 billion, about 90 million to about 1.1 billion, about 1 billion to 1.25 billion, about 1.25 billion to about 1.5 billion, and ranges including and/or spanning the aforementioned values. Without being bound to a particular theory, when injected, it is believed that CDCs are transient residents in the subject. Depending on the embodiment, the degree of CDC retention varies. For example, in several embodiments, the retention rate is between about 0.01% and 10%, including about 0.01% to about 0.05%, about 0.05% to about 0.1%, about 0.1% to about 0.5%, about 0.5% to about 1.0%, about 1.0% to about 2.5%, about 2.5% to about 5%, about 5% to about 10%, and ranges including and/or spanning the aforementioned values. Thus, in some embodiments, the equivalent amount of CDC-XOs delivered to the patient is calculated as the amount of CDC-XOs that would be released via an administration (e.g., injection or infusion) of the disclosed amounts CDCs over a given time of CDC residence in the body of about 1 week, about 2 weeks, about 3 weeks, or more. In certain instances, the dosage may be prorated to body weight (range 100,000-1 M CDCs/kg body weight total CDC dose). In some embodiments, for injection into the heart, the number of administered CDCs includes 25 million CDCs per coronary artery (i.e., 75 million CDCs total) as another baseline for exosome (or XO) or extracellular vesicle (EV) dosage quantity.

In several embodiments, CDC and/or CDC-derived exosomes (or CDC-XOs) are administered based on a ratio of the number of exosomes the number of cells in a particular target tissue, for example CDC or CDC-XO:target cell ratio ranging from about 10⁹:1 to about 1:1, including about 10⁸:1, about 10⁷:1, about 10⁶:1, about 10⁵:1, about 10⁴:1, about 10³:1, about 10²:1, about 10:1, and ratios in between these ratios. In additional embodiments, CDC and/or CDC-derived exosomes are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the CDC and/or CDC-derived exosomes are to be administered in conjunction with the concurrent therapy (e.g., cells that can still shed exosomes, pharmaceutical therapy, nucleic acid therapy, and the like) the dose of CDC and/or CDC-derived exosomes administered can be adjusted accordingly (e.g., increased or decreased as needed to achieve the desired therapeutic effect).

In some embodiments, the CDC and/or CDC-XO quantity delivered to the patient (e.g., the dose) may be measured by weight (in mg) of CDCs and/or CDC-XOs (e.g., where the solution and/or milieu surrounding the CDCs and/or CDC-XOs has been removed or substantially removed). For instance, in some embodiments, a dose of CDCs and/or CDC-XOs may comprise equal to or at least about the following weights in mg: about 0.001 to about 0.005, about 0.005 to about 0.01, about 0.01 to about 0.05, about 0.05 to about 0.1, about 0.1 to about 0.5, about 0.5 to about 1, about 1 to about 10, about 10 to about 25, about 25 to about 50, about 50 to about 75, about 75 to about 100, or ranges including and/or spanning the aforementioned values. As discussed in additional detail herein, those masses are representative, of the number of CDCs and/or CDC-XOs that are dosed to a subject, depending on the embodiment. For example, in several embodiments, the number of CDCs in a dose can range from about 5×10⁴ to about 2×10⁹, including about 5×10⁴ to about 1×10⁵, about 1×10⁵ to about 2.5×10⁵, about 2.5×10⁵ to about 1×10⁶, about 1×10⁶ to about 1×10⁷, about 1×10⁷ to about 1×10⁸, about 1×10⁸ to about 1×10⁹, about 1×10⁹ to about 2×10⁹, about 2×10⁹ to about 5×10⁹, and ranges including and/or spanning the aforementioned values. Likewise, depending on the embodiment, the number of exosomes or particles (e.g., vesicles) dosed to a subject can range from about 1×10⁹ to about 2×10¹⁴, including about 1×10⁹ to about 2×10⁹, about 2×10⁹ to about 4×10⁹, about 4×10⁹ to about 1×10¹⁰, about 1×10¹⁰ to about 1×10¹¹, about 1×10¹¹ to about 1×10¹², about 1×10¹² to about 2×10¹², about 2×10¹² to about 2×10¹³, about 2×10¹³ to about 1×10¹⁴, about 1×10¹⁴ to about 2×10¹⁴, and ranges including and/or spanning the aforementioned values. In some embodiments, the CDC and/or CDC-XO quantity delivered to the patient may be measured by protein weight (in mg) and/or by total cell or vesicle weight (e.g., where water has been removed from the area outside the cells or vesicles). In some embodiments, the CDC and/or CDC-XO quantity delivered to the patient is equal to 1-10, 10-25, 25-50, 50-75, 75-100, or 100 or more mg protein. In some embodiments, administering a therapeutically effective amount of a composition includes about 1 to about 100 mg XO protein in a single dose.

In several embodiments, the methods include administering any suitable number of doses of the CDCs and/or CDC-derived exosomes to the subject. In some embodiments, a single dose is administered in two or more separate batches. In several embodiments, the CDCs and/or CDC-derived exosomes are delivered in a single, bolus dose. In some embodiments, however, multiple doses of CDCs and/or CDC-derived exosomes may be delivered. In certain embodiments, CDCs and/or CDC-derived exosomes can be infused (or otherwise delivered) at a specified rate over time. In several embodiments, when CDCs and/or CDC-derived exosomes are administered within a relatively short time frame after an adverse event (e.g., onset of a cytokine storm or ARDS), their administration prevents the generation or progression of damage to a target tissue. For example, if CDCs and/or CDC-derived exosomes are administered within about 20 to about 30 minutes, within about 30 to about 40 minutes, within about 40 to about 50 minutes, within about 50 to about 60 minutes post-adverse event, the damage or adverse impact on a tissue is reduced (as compared to tissues that were not treated at such early time points). In some embodiments, the administration is as soon as possible after an adverse event. In some embodiments the administration is as soon as practicable after an adverse event (e.g., once a subject has been stabilized in other respects). In several embodiments, administration is within about 1 to about 2 hours, within about 2 to about 3 hours, within about 3 to about 4 hours, within about 4 to about 5 hours, within about 5 to about 6 hours, within about 6 to about 8 hours, within about 8 to about 10 hours, within about 10 to about 12 hours, and overlapping ranges thereof. Administration at time points that occur longer after an adverse event are effective at preventing damage to tissue, in certain additional embodiments.

In some embodiments, the CDCs and/or CDC-derived exosomes are administered once, e.g., in a single dose or in a single treatment episode, to the subject. In some embodiments, the CDCs and/or CDC-derived exosomes are administered chronically. In some embodiments, the CDCs and/or CDC-derived exosomes are administered 1, 2, 3, 4, 5, 6, 7, 8, 9, 10, 12, 15, 20, 25, 30, 40 or 50 or more times, or any number of times in between those listed, to the subject. The CDCs and/or CDC-derived exosomes can be administered at any suitable interval between consecutive doses. In some embodiments, the interval between administering consecutive doses of CDCs and/or CDC-derived exosomes is about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or about 6 months, or any interval defined by any two of the aforementioned lengths of time. In some embodiments, the CDCs and/or CDC-derived exosomes are administered to the subject weekly, bi-weekly, monthly, bi-monthly, semi-annually or annually. In some embodiments, the CDCs and/or CDC-derived exosomes are administered continuously. In certain embodiments, the CDCs and/or CDC-derived exosomes are administered continuously to the subject for 1 hour or more, e.g., 3 hours or more, 6 hours or more, 12 hours or more, 1 day or more, 5 days or more, 2 weeks or more, including 1 month or more, or any period of time in between the values listed.

Described herein are compositions and methods providing significant benefits in the repair or regeneration of damaged or diseased tissues via CDCs and CDC-XOs. Certain supporting techniques are described in, for example, U.S. App. Ser. Nos. 11/666,685, 12/622,143, 12/622,106, 14/421,355, PCT App. No. PCT/US2013/054732, PCT/US2015/053853, PCT/US2015/054301 and PCT/US2016/035561, which are fully incorporated by reference herein.

### Proposed Clinical Administration

The following is a non-limiting example of a proposed administration and evalution program for CDCs to treat viral infection.

Subjects with confirmed COVID-19 infection and who are in critical condition as indicated by life support measurements will undergo intravenous administration of allogeneic CDCs. For this specific protocol subjects must meet the following inclusion criteria for enrollment:
Male or female subjects at least 18 years of age at time of consent
Confirmed COVID-19 infection
In critical condition as indicated by life support measurements
Have one or more of the following laboratory parameters:
lymphocytopenia,
elevated IL-6,
elevated Troponin I/Troponin T (TnI/T),
elevated myoglobin,
elevated C-Reactive Protein (CRP).

According to additional embodiments, CDCs (or CDC-derived exosomes) are administered to subjects exhibiting different, fewer or less severe symptoms than those listed above. CDC infusions will be given on Day 1 and weekly up to a maximum of 4 doses, based on clinical course. Prior to the second CDC administration only, medications may be administered to the subject at the Investigator's discretion based on the pre-treatment guidelines in Table 1 and/or institutional protocols to minimize the risk of potential severe allergic reactions such as anaphylaxis. Final decisions regarding the medication(s), dose(s) administered, and route(s) of administration will be determined by the Investigator taking into consideration the subject's medical status, COVID-19 related conditions, concomitant medications, and medical history. For any pre-treatment medication administered, the FDA approved label will be reviewed for information on potential side effects and/or drug interactions and followed for detailed instructions on weight-based dosing.

TABLE 1
Pre-treatment Guidelines

| | | | Timing of | |
|---|---|---|---|---|
| | | | Administration | |
| Medicationⁱ⁾ | Dose | Route | Prior to Infusion | |
| Prednisone | 1 mg/kg | PO | 12-14 | hours |
| or | (up to 60 mg max) | PO | | |
| Methylprednisolone | 0.8-1.2 mg/kg | | | |
| | (up to 50 mg max) | | | |
| Prednisone | 1 mg/kg | PO | 2-3 | hours |
| or | (up to 60 mg max) | PO | | |
| Methylprednisolone | 0.8-1.2 mg/kg | | | |
| | (up to 50 mg max) | | | |
| Diphenhydramine | 1 mg/kg | IV | 1 | hour |
| or | (up to 25 mg) | PO | | |
| Cetirizine | 10 mg | | | |
| (if ≥6 yrs old) | | | | |
| Ranitidine | 1-2 mg/kg | IV | 1 | hour |
| | (up to 75 mg max) | PO | | |
| | or | | | |
| | 75 mg | | | |

Currently no validated treatment options are available for patients with this condition. Allogeneic CDCs have been shown to preserve function and protect against repolarization abnormalities in experimental myocarditis, polarize macrophages to a healing phenotype, mitigate cardiac ischemic injury, attenuate fibrosis in heart and kidney in various preclinical models, attenuate inflammatory cytokines in the mdx mouse muscular dystrophy model and have demonstrated bioactivity in a variety of inflammatory disease models (see enclosed preclinical data summary and rationale). The pathophysiology of COVID-19 involves epithelial injury and destruction, direct infection of leukocytes and immune response to injury/infection. The safety of allogeneic CDCs has been shown in clinical trials of myocardial infarction, dilated cardiomyopathy, Duchenne muscular dystrophy and post-MI cardiac dysfunction.

The safety of multiple infusions of CDCs has been evaluated in previous clinical trials included in IND 17800. With the exception of severe allergic reactions, which occurred after multiple administrations of CDCs, no safety signals were observed in two clinical trials that evaluated multiple administrations of CDCs. Such reactions may occur after multiple administrations of cell products and can be successfully mitigated or prevented with established pre-treatment regimens.

The patient will be observed during the lengths of hospitalization and monitored for outcome and safety with vital signs, physical examinations, ECGs, PFTs, clinical laboratory testing (including CBC, BMP, BNP, CRP, ESR, hsCRP, cytokine assay, viral load/nasal swab), troponin I/troponin T and transthoracic echocardiogram. Additional CT and/or cardiac MRI imaging may be performed, as appropriate. Safety and outcome data (including mortality, need for additional levels of supportive care, length of stay, etc.) will be collected and reported in a written summary to FDA at the conclusion of treatment and follow-up. Additional samples of blood may be collected for proteomic analysis.

Subjects will receive a total dose of 150 million allogeneic CDCs per administration. Drug product frozen concentrate is supplied in a volume of 10 mL of cryogenic cell preservation solution consisting of 50% (v/v) CryoStor® CS10 containing 10% DMSO, 40% (v/v) HypoThermosol®, and 10% (v/v) Albumin (Human) 25%. The primary container is a Daikyo Crystal Zenith® vial with chlorobutyl stopper that is shipped and stored at less than or equal to −140 ° C. until thawed for use. Each unit of drug product contains 7.5×10⁷ (75 million or 75M) CDCs (formulated with 20% cell overage to ensure 75M cell dose for administration). Two units make up the total dose used in this non-limiting embodiment of a study for each infusion of 150M CDCs.

Once thawed, each vial of CDCs is diluted with 40 mL of 5% human serum albumin (HSA) for IV use only, containing the albumin component of human blood, such as Octapharma brand, in a 60-mL syringe. CDC, in this study, are intended for parenteral (IV) route of administration via a peripheral access device (or central venous access device if a central line or portacath was placed for clinical indications and is indwelling in a subject at the time of randomization). Prior to the CDC infusion, the IV line will be flushed with a bolus of 5% HSA (approximately 4 mL over 2 minutes±30 seconds). The final ready-to-use CDC dose in a total volume of 50 mL is infused with a commercially available syringe pump (510(k) cleared for human use) set at an initial rate of 1 mL/min for 10 minutes (±30 seconds). If no signs or symptoms of a potential allergic reaction are observed, the infusion will continue at a rate of 4 mL/min for a total infusion time of 20 minutes (±2 minutes). Upon completion of the first CDC infusion and prior to the second infusion, residual material is washed out of the IV tubing with a bolus of 5% HSA (approximately 4 mL over 2 minutes±30 seconds). The next CDC dose in the second 60 mL syringe is administered to the subject at a rate of 4 mL/min, for a total infusion time of 12.5 (±1 minute). After the second infusion, residual material is washed out of the IV tubing with a bolus of 5% HSA (approximately 4 mL over 2 minutes ±30 seconds).

### EXAMPLES

### Example 1

The following non-limiting methods were used for Example 2.

A study to evaluate safety and impact of administration of allogeneic CDCs in critically ill COVID-19 patients was conducted at Cedars-Sinai Medical Center (CSMC), Los Angeles, Calif., USA. Each individual case study was approved by the Institutional Review Board at CSMC, and each patient (or their legal representative) gave written informed consent. Likewise, each case was reviewed in detail with the US Food and Drug Administration (FDA) to obtain approval for compassionate administration of CAP-1002 under Emergency Use protocols. Patients

Patients were evaluated for possible recruitment if they met the following criteria: (1) laboratory confirmed COVID-19, diagnosed using a reverse transcriptase polymerase chain reaction (RT-PCR) assay; (2) severe COVID-19, with respiratory failure requiring increasing supplemental oxygen and/or shock requiring inotropes; (3) not enrolled in another clinical trial of an experimental agent for COVID-19; and (4) ability of the patient (or legally authorized representative) to provide informed consent. Patients received adjunctive therapy (including hydroxychloroquine and tocilizumab) per clinical practice protocols then in place for COVID-19 at CSMC. Lack of clinical improvement or deterioration despite standard care was the primary reason to evaluate patients for emergent administration of allogeneic CDCs. Exclusion criteria included known hypersensitivity to dimethyl sulfoxide (DMSO; a component of CAP-1002), prior stem cell therapy, pre-existing terminal illness (e.g., metastatic cancer), need for mechanical circulatory support and dialysis. In general, patients with multi-organ failure who were deemed to be too sick for any intervention were excluded from the study.

### Contemporaneous Control Group

Given the compassionate use nature of the case series, there was no randomization. Nevertheless, a basis for comparison of outcomes and clinical characteristics between the 6 CAP-1002-treated subjects reported here, and other critically ill patients simultaneously hospitalized for COVID-19 at CSMC, with similar baseline characteristics was sought. A set of patients who were admitted to CSMC with RT-PCR confirmed SARS-CoV-2 infection, who required mechanical ventilation were retrospectively characterized. To render the comparison as fair as possible, clinical status at 30.7 days of hospitalization (to match the average in the CAP-1002-treated subjects) was quantified and the analysis was limited to patients who received anti-IL6 or anti-IL6 receptor agents during the hospitalization as standard-of-care therapy (as did all of the CAP-1002-treated subjects). Patients were excluded if they: (1) did not have at least 30.7 days of follow-up from admission to the terminal event (death or hospital discharge); (2) were enrolled in any clinical trial requiring informed consent; and (3) had a tracheostomy placed prior to the current admission.

### Cell Manufacturing

CAP-1002 was manufactured by Capricor as described previously (Chakravarty et al. (2017) Cell Transplant 26:205-214; Smith et al. (2007) Circulation 115:896-908, both incorporated by reference herein in their entirety). Based on the IV infusion protocol and dose of CAP-1002 used in the HOPE-2 clinical trial of Duchenne muscular dystrophy (NCT03406780), a dose of 150 million allogeneic CDCs was selected to administer in this study, though as discussed above, other doses are used in some embodiments. Cell product frozen concentrate was supplied in a volume of 10 ml of cryogenic cell preservation solution consisting of 50% (v/v) CryoStor® CS10 containing 10% DMSO, 40% (v/v) HypoThermosol®, and 10% (v/v) Albumin (Human, HSA) 25%. The primary container was a Daikyo Crystal Zenith® vial with chlorobutyl stopper that was stored at less than or equal to −140 ° C. until thawed for use. Once thawed, each vial of CAP-1002 was diluted with 40 ml of 5% HSA for IV use only, containing the albumin component of human blood in a 60-ml syringe.

### Cell Administration

CAP-1002 was administered IV via a peripheral access device (or a central venous access device, if it was available at the time of administration). Following administration of the cells, the patients were monitored for signs of clinical and biochemical improvement (or deterioration). If the patients continued to require significant supplemental oxygen after one week, they were re-evaluated for a second dose of cell administration as a booster dose. Prior to administration of the second dose, patients were premedicated with an antihistamine to mitigate any potential allergic reactions (which, though clinically insignificant, were noted in two patients in the HOPE-2 clinical trial when premedication was omitted before the second dose). The rationale for a second administration of CAP-1002 is based on preclinical evidence demonstrating additional benefit with repeat dosing of CDCs. Importantly, a repeat administration of CDCs was not associated with sensitization or adverse immunologic responses in rodents.

The final ready-to-use cell product in a total volume of 50 ml was infused with a commercially available syringe pump [510(k) cleared for human use] set at an initial rate of 1 ml/min for 10 min (±30 s). If no signs or symptoms of a potential adverse reaction were observed, the infusion was continued at a rate of 4 ml/min for a total infusion time of 20 min (±2 min). Upon completion of infusion of the first vial of CAP-1002, and prior to infusion of the second vial, residual material was washed out of the IV tubing with a bolus of 5% HSA (approximately 4 ml over 2 min±30 s). The next cell dose of 75 million cells in the second 60-ml syringe was administered to the subject at a rate of 4 ml/ min, for a total infusion time of 12.5 (±1 min). After the second infusion, residual material was washed out of the IV tubing with a bolus of 5% HSA (approximately 4 ml over 2 min±30 s).

### RT-PCR

RT-PCR was performed on nasopharyngeal specimens collected from patients who met Centers for Disease Control and Prevention clinical and/or epidemiological criteria for COVID-19 testing. If RNA from SARS-CoV-2 was detected, the test was positive and the patient was considered infected with virus and presumed to be contagious. Laboratory test results were considered in the context of clinical observations and epidemiologic data in making a final diagnosis and patient management decisions. The test was developed and its performance characteristics were determined by CSMC Department of Pathology and Laboratory Medicine. The laboratory is certified under the Clinical Laboratory Improvement Amendments (CLIA) as qualified to perform high-complexity clinical laboratory testing. This test was validated, but independent review by FDA of this validation is pending.

### Statistical Analysis

Pooled data are presented as means±standard deviation (SD), range, and median. Due to small sample size, statistical tests comparing CAP-1002 and control groups were not performed.

### Example 2

This non-limiting example shows safety of administering allogeneic CDCs to severe COVID-19 patients.

### Overview

There are no definitive therapies for patients with severe acute respiratory syndrome coronavirus-2 (SARS-CoV-2) infection. Therefore, new therapeutic strategies are needed to improve clinical outcomes, particularly in patients with severe disease. This case series explores the safety and effectiveness of intravenous allogeneic cardiosphere-derived cells (CDCs), formulated as CAP-1002, in critically ill patients with confirmed coronavirus disease 2019 (COVID-19). Adverse reactions to CAP-1002, clinical status on the World Health Organization (WHO) ordinal scale, and changes in pro-inflammatory biomarkers and leukocyte counts were analyzed. All patients (n=6; age range 19-75 years, 1 female) required ventilatory support (invasive mechanical ventilation, n=5) with PaO₂/FiO₂ ranging from 69 to 198. No adverse events related to CAP-1002 administration were observed. Four patients (67%) were weaned from respiratory support and discharged from the hospital. One patient remained mechanically ventilated; all survived. A contemporaneous control group of critically ill COVID-19 patients (n=34) showed 18% overall mortality at a similar stage of hospitalization. Ferritin was elevated in all patients at baseline (range of all patients 605.43-2991.52 ng/ml) and decreased in 5/6 patients (range of all patients 252.89-1029.90 ng/ml). Absolute lymphocyte counts were low in 5/6 patients at baseline (range 0.26-0.82×103/μl) but had increased in three of these five patients at last follow-up (range 0.23-1.02×103/μl). In this series of six critically ill COVID-19 patients, intravenous infusion of CAP-1002 was well tolerated and associated with resolution of critical illness in 4 patients. This series demonstrates the apparent safety of CAP-1002 in COVID-19.

### Results

Six patients (age range 19-75 years, one woman) underwent IV infusion of CAP-1002 containing 150 million allogeneic CDCs. Consistent with the Berlin Criteria, all patients had acute respiratory distress syndrome (ARDS) prior to CAP-1002 infusion, with decreased PaO₂/FiO₂ ratios (range 69.0-198.0; median 142.5), diffuse bilateral pulmonary infiltrates on chest imaging (n=6, 100%), and evidence of preserved cardiac function on transthoracic echocardiography (LVEF range 50-75%; median 55%), suggesting a non-cardiac origin of respiratory failure. Sequential organ failure and assessment (SOFA) scores ranged from 2 to 8 prior to cell administration. All six patients had received an anti-IL6 agent, tocilizumab, prior to the CAP-1002 infusion. In addition to tocilizumab, patient 1 received lopinavir/ritonavir and the remaining patients received hydroxychloroquine for 5 days prior to the cell infusion. Patient 2 was 19 years old, but was morbidly obese (BMI 42.2). Patients 1, 3, 4, 5 had significant cardiovascular comorbidities (Table 1 in FIG. 36). All patients were intubated (n=5) or required high-flow nasal cannula (HFNC) support (n=1) at the time of enrollment. The one patient receiving HFNC support was deemed high risk for imminent intubation. The remaining intubated patients had been on invasive mechanical ventilation for 2-11 days prior to cell administration. The first patient was intubated at the time of enrollment, but extubated by the time of cell administration. This patient was determined to be at a high risk for reintubation given high oxygen requirements on HFNC and, therefore, underwent cell administration.

Blood cell counts, inflammatory markers and cytokine levels are summarized in Table 2 (in FIG. 37) and FIGS. 38A-38D. All patients had elevated CRP (FIG. 38A), ferritin (FIG. 38B), IL-6 (FIG. 38C) and TNFα prior to cell infusion. Five patients had lymphopenia prior to infusion (FIG. 38D). IL1α and IL-1β levels were not elevated in this cohort. Ferritin and CRP levels decreased in 5 out of 6 patients following cell infusion (FIGS. 38B and 38A, respectively). IL-6 levels were increased in all six patients at baseline and decreased in four patients (FIG. 38C). IL-10 levels remained below reference range in one patient, decreased in three patients, and increased in two patients (Table 2 in FIG. 37). Upon admission, two patients had mildly elevated cardiac troponin I levels (range of all patients<0.02-0.07 ng/ml; median 0.01 ng/ml). During the course of the hospitalization, cardiac troponin I levels increased in 4 patients (range of all patients<0.02-1.26 ng/ml; median 0.13 ng/ml) within 4-16 days of admission, but subsequently decreased in all these patients (range of all patients<0.02-0.15 ng/ ml; median 0.07 ng/ml). Similarly, D-dimer levels were mildly elevated in four patients upon admission (range of all patients 0.34-2.22 μg/ml; median 0.83 μg/ml), increased in five patients within 4-17 days of admission (range of all patients 5.36-20.00 μg/ml; median 20.00 μg/ml), and subsequently decreased in four patients (range of all patients 1.53-20.00 μg/ml; median 2.45 μg/ml).

The clinical course of individual patients is summarized in FIG. 39. No patient experienced any complications related to CAP-1002 administration. Patients 1 and 4 received a second dose of CAP-1002, after pretreatment with diphenhydramine, likewise with no complications. All intubated patients improved clinically after cell infusion, except patients 4 and 6, who, at the time of this summary study, were still in ICU but remain stable. Patient 1 was already extubated at the time of infusion, and CAP-1002 was administered due to rising oxygen requirements (on HFNC) and concern for reintubation. Similarly, patient 6 received the infusion in the setting of respiratory deterioration and imminent risk for intubation. 5 days post-infusion, patient 6 had not been intubated. Patients 2, 3 and 5 were extubated on post-infusion days 3, 4 and 1, respectively. Except for patients 4 and 6, all patients were transferred out of the ICU and discharged from the hospital (Table 1 in FIG. 36). There were no overt instances of acute stroke, acute myocardial infarction, or acute pulmonary embolism during the hospitalization at the time of last follow-up. One patient (patient #4) received additional antibiotics for a possible bacterial superinfection, although the diagnosis remains presumptive and no source has been identified. All patients were alive at the end of the study, with a mean follow-up of 13.5±4.6 days. This is in contrast to 6 deaths (18%) noted among 34 similar patients who received treatment for COVID-19 in the medical ICU around the same time (notably all patients were treated with anti-IL6 therapy; Table 3 in FIG. 40).

In some embodiments, administering a therapeutically effective amount of CDCs to a severe COVID-19 patient is safely tolerated by the patient. In some embodiments, administering a clinically relevant amount of CDCs to a severe COVID-19 patient improves the clinical status of the patient. In some embodiments, administering a clinically relevant amount of CDCs to a severe COVID-19 patient reduces inflammatory markers and cytokine levels in the patient. In some embodiments, administering a clinically relevant amount of CDCs to a severe COVID-19 patient reduces the duration of intubation, promotes extubation, reduces a likelihood of the need for intubation, and/or prevents intubation.

### Example 3

This non-limiting example summarizes the results of Example 2.

Administration of CAP-1002 as a compassionate therapy for patients with severe COVID-19 and significant comorbidities was safe, well tolerated without serious adverse events, and associated with clinical improvement, as evidenced by extubation (or prevention of intubation). All the critically ill patients who received CAP-1002 survived, and four out of six have been discharged. This is in contrast to high mortality rates (˜50%) reported for critically ill patients with COVID-19. An age- and gender-matched retrospectively assembled cohort of COVID-19 patients also showed higher mortality (6 of 34 patients) compared to the compassionate-use series (0 of 6), but statistical comparisons were not attempted given the small number of CAP-1002-treated patients. Most patients receiving CAP-1002 also showed improvements in inflammatory markers, though to varying degrees. Similar to other COVID-19 cohorts, the patients exhibited elevated cardiac troponin I and D-dimer levels. These biomarkers, however, decreased in all but 1 of the patients at the date of last follow-up.

The underlying pathophysiology of COVID-19 involves a maladaptive immune response to SARS-CoV-2 infection with increased levels of IL-6, IL-10, IL-2 and TNFα produced by macrophages, and fewer CD4+and CD8+T cells, but no significant changes in B-cell counts. The dysregulated immune function with cytokine storm leads to lung, heart, and other end-organ injury. Extensive preclinical and some clinical studies suggest that cell therapy may attenuate inflammation.

CDCs are stromal progenitor cells isolated from human heart tissue through well-specified culture techniques and exert their effects in a paracrine manner by secreting exosomes (nanosized vesicles with bioactive payload). CDCs target multiple cytokine pathways (e.g., TNFα, IFN-γ, IL-1β, IL-6) that are associated with disease progression and poor outcomes in COVID-19 (FIG. 4). For example, CDCs have the capacity to polarize macrophages toward an anti-inflammatory and healing phenotype. These anti-inflammatory effects have been demonstrated in animal models of myocardial ischemia, myocarditis, muscular dystrophy, aging, heart failure with preserved ejection fraction, pulmonary arterial hypertension and dilated cardiomyopathy. Finally, based on preclinical work, a majority of IV CDCs are retained in the lungs.

A number of issues pertinent to use of cell therapy in SARS-CoV-2 infection may be explored. These include the optimal sources of cells and dosing strategies. With respect to safety, no serious adverse events have been noted with administration of CAP-1002 or MSCs from various sources in patients. Another issue related to safety is the potential secretion of IL-6 by CDCs. This potential source of IL-6, however, appears to be negligible, as multiple preclinical models demonstrated the opposite effect following CDC administration: significantly decreased serum levels of IL-6 after CDC infusion. Thus, future studies may focus on recognized endpoints including overall mortality, survival to hospital discharge and beyond, length of hospital stay and ventilator-free days, with randomization and rigorous controls. A composite ordinal scale reflecting improvement in COVID-19 disease state may trenchantly capture the key clinical outcomes (as recommended by the WHO R&D Blueprint).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

What is claimed is:

1. A method of treating a respiratory viral infection, comprising administering to a subject in need of treating a respiratory viral infection, or one or more symptoms or sequelae thereof, a therapeutically effective amount of cardiosphere-derived cells (CDCs) and/or CDC-derived exosomes, thereby treating the viral infection or one or more symptoms or sequelae thereof.

2. The method of claim 1, wherein the respiratory viral infection is COVID-19.

3. The method of claim 2, wherein the subject has severe COVID-19.

4. The method of claim 3, wherein the subject has acute myocarditis, acute respiratory distress syndrome (ARDS) and/or hypoxemia.

5. The method of claim 3, wherein the subject is on ventilatory support and/or is in an intensive care unit (ICU).

6. The method of claim 5, wherein the subject is intubated.

7. The method of claim 2, wherein the subject has elevated levels of one or more inflammatory markers or cytokines, and/or has lymphopenea.

8. The method of claim 7, wherein the one or more inflammatory markers or cytokines comprise one or more of ferritin, CRP, IL-6, and TN Fα.

9. The method of claim 1, wherein administering the therapeutically effective amount of CDCs and/or CDC-derived exosomes reduces a hyperinflammatory response, or sequelae thereof, associated with a SARS-CoV-2 infection.

10. The method of claim 2, wherein administering the therapeutically effective amount of CDCs and/or CDC-derived exosomes reduces one or more of respiratory distress, myocardial injury, and/or inflammatory cytokine level.

11. The method of claim 2, wherein administration of the therapeutically effective amount of CDCs and/or CDC-derived exosomes reduces length of hospital stay, length of stay in an ICU, or duration of ventilatory support.

12. The method of claim 2, wherein the therapeutically effective amount of CDCs comprises from about 1×10⁷ to about 1×10⁹ cells.

13. The method of claim 2, wherein the therapeutically effective amount of CDC-derived exosomes comprises from about 2×10⁸ to about 2×10¹⁰ exosomes.

14. The method of any one of the preceding claims, further comprising administering to the subject at least one additional dose of the therapeutically effective amount of cardiosphere-derived cells (CDCs) and/or CDC-derived exosomes.

15. The method of claim 14, wherein the at least one additional dose is administered on about a weekly basis.

16. The method of claim any one of the preceding claims, further comprising administering to the subject at least one additional therapeutic, wherein the additional therapeutic is selected from an antiviral agent; an analgesic agent; anti-arthritic agent; anti-asthmatic agent; anticholinergic agent;
antihistamine; anti-infective agent; anti-inflammatory agent; cardiovascular medicament; gastrointestinal medicament; immunosuppressive agent; leukotriene inhibitor; narcotic agonist or antagonist; peptide drug;
phytonutrient; vasodilator; vitamins or mineral supplement, or a combination thereof.

17. The method of claim 16, wherein the at least one additional therapeutic comprises one or more of: clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine, promethazine, diphenhydramine, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazine, terfenadine, chlorpheniramine, dimenhydrinate, fexofenadine, orphenadrine, pheniramine, cimetidine, famotidine, lafutidine, nizatidine, ranitidine, roxatidine, cortisone hydrocortisone, hydrocortisone-21-monoester, hydrocortisone-17,21-diesters, alclometasone, dexamethasone, flumethasone, prednisolone, methylprednisolone, betamethasone, fluocinonide, prednisone, triamcinolone, methotrexate, leflunomide, cyclophosphamide, azathioprine, celecoxib, rofecoxib, a soluble cytokine receptor, anti-cytokine antibody, etanercept, infliximab, cyclosporin, tacrolimus, and rapamycin.

18. The method of any one of the preceding claims, further comprising administering to the subject an anti-viral therapy.

19. A method of treating a respiratory viral infection, comprising administering to a subject in need of treating a respiratory viral infection, or one or more symptoms or sequelae thereof, a therapeutically effective amount of cardiosphere-derived cells (CDCs), thereby treating the respiratory viral infection or one or more symptoms or sequelae thereof,
wherein the respiratory viral infection is COVID-19, wherein the therapeutically effective amount comprises from about 1×10⁷ to about 1×10⁹ CDCs, wherein administration of the therapeutically effective amount of CDCs reduces a hyperinflammatory response, or a sequelae thereof, associated with a SARS-CoV-2 infection.

20. A method of treating a respiratory viral infection, comprising:
administering to a subject in need of treating a respiratory viral infection, or one or more symptoms or sequelae thereof, a population of cardiosphere-derived cells (CDCs) and/or CDC-derived exosomes, wherein the population comprises about 1×10⁸ to about 2×10⁸ CDCs and/or about 2×10⁸ to about 2×10¹⁰ CDC-derived exosomes; and
administering to the subject at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof;
wherein the administration of the CDCs and/or CDC-derived exosomes and the at least one additional therapeutic reduce one or more of respiratory distress, myocardial injury, and/or inflammatory cytokine level, thereby treating the respiratory viral infection or one or more symptoms or sequelae thereof.

21. The method of claim 20, wherein the respiratory viral infection is COVID-19.

22. The method of claim 21, wherein the subject has severe COVID-19.

23. The method of claim 22, wherein the subject has acute respiratory distress syndrome (ARDS) and/or acute myocarditis.

24. The method of claim 22, wherein the subject is on ventilatory support and/or is in an intensive care unit (ICU).

25. The method of claim 21, wherein the subject has elevated levels of one or more inflammatory markers or cytokines, and/or has lymphopenea.

26. The method of claim 20, further comprising administering to the subject at least one additional dose of the population of CDCs and/or CDC-derived exosomes.

27. The method of claim 26, wherein the at least one additional dose is administered on about a weekly basis.

28. The method of any one of claims 20 to 27, wherein the antihistamine is selected from clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine, promethazine, diphenhydramine, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazine, terfenadine, chlorpheniramine, dimenhydrinate, fexofenadine, orphenadrine, pheniramine, cimetidine, famotidine, lafutidine, nizatidine, ranitidine, and roxatidine, or a combination thereof.

29. The method of any one of claims 20 to 28, wherein the anti-inflammatory agent is a corticosteroid.

30. The method of claim 29, wherein the anti-inflammatory agent is selected from cortisone hydrocortisone, hydrocortisone-21-monoester, hydrocortisone-17,21-diester, alclometasone, dexamethasone, flumethasone, prednisolone, methylprednisolone, betamethasone, fluocinonide, prednisone, triamcinolone, or a combination thereof.

31. The method of any one of claims 20 to 30, wherein the immunosuppressive agent is methotrexate, leflunomide, cyclophosphamide, azathioprine, or a combination thereof.

32. The method of any one of claims 20 to 31, wherein the cyclooxygenase-2 inhibitor is celecoxib, rofecoxib, or a combination thereof.

33. The method of any one of claims 20 to 32, wherein the cytokine inhibitor is a soluble cytokine receptor, anti-cytokine antibody, or a combination thereof.

34. The method of any one of claims 20 to 33, wherein the TNF-α inhibitor is etanercept, infliximab, or a combination thereof.

35. The method of any one of claims 20 to 34, wherein the additional therapeutic is cyclosporin, tacrolimus, rapamycin, or a combination thereof.

36. The method of any one of the preceding claims, wherein the CDCs are allogeneic CDCs.

37. The method of any one of the preceding claims, wherein the exosomes are allogeneic CDC-derived exosomes.

38. The method of any one of the preceding claims, wherein the administering comprises parenteral administration of the population of CDCs and/or CDC-derived exosomes.

39. The method of any one of the preceding claims, wherein the population of CDCs and/or CDC-derived exosomes is administered intravenously.

40. The method of any one of the preceding claims, wherein the administering comprises infusing or injecting the population of CDCs and/or CDC-derived exosomes.

41. The method of any one of the preceding claims, wherein the administering comprises administering the CDCs.

42. The method of any one of the preceding claims, wherein the administering comprises administering the CDC-derived exosomes.

43. The method of any one of the preceding claims, wherein the subject has one or more comorbidities.

44. The method of claim 43, wherein the subject has a cardiorespiratory dysfunction.

45. The method of claim 43, wherein the subject has hypertension, diabetes, obesity, and/or coronary heart disease.

46. Use of a therapeutically effective amount of CDCs and/or CDC-derived exosomes, and optionally at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof, for the treatment of a respiratory viral infection.

47. Use of a therapeutically effective amount of CDCs and/or CDC-derived exosomes, and optionally at least one additional therapeutic, wherein the additional therapeutic is selected from an antiviral agent, an antihistamine, anti-inflammatory agent, immunosuppressive agent, cyclooxygenase-2 inhibitor, TNF-α inhibitor, cytokine inhibitor or a combination thereof, in the manufacture of a medicament for the treatment of a respiratory viral infection.

48. The use of claim 46 or 47, wherein the respiratory viral infection is COVID-19.

49. The use of claim 48, wherein the respiratory viral infection is severe COVID-19.

50. The use of any one of claims 46-49, wherein the therapeutically effective amount of CDCs comprises from about 1×10⁷ to about 1×10⁹ cells.

51. The use of any one of claims 46-49, wherein the therapeutically effective amount of CDC-derived exosomes comprises from about 2×10⁸ to about 2×10¹⁰ exosomes.
